(19) United States
(12) Patent Application Publication
(10) Pub. No.: US 2021/0361600 A1
(43) Pub. Date: Nov. 25, 2021

(54) METHODS FOR INDUCING CHONDROGENESIS

(71) Applicant: THE SCRIPPS RESEARCH INSTITUTE, La Jolla, CA (US)

(72) Inventors: Peter G. SCHULTZ, La Jolla, CA (US); Arnab K. CHATTERJEE, San Diego, CA (US); Timothy M. WRIGHT, Rancho Santa Fe, CA (US); John WISLER, Carlsbad, CA (US); Vadim KLYUSHNICHENKO, Carsbad, CA (US)

(21) Appl. No.: 16/606,672

(22) PCT Filed: Apr. 23, 2018
(86) PCT No.: PCT/US2018/028939

§ 371 Date: Oct. 18, 2019

Related U.S. Application Data
(60) Provisional application No. 62/489,397, filed on Apr. 24, 2017.

(51) Int. Cl. A61K 31/18; A61P 19/02

(52) U.S. Cl. CPC A61K 31/18; A61P 19/02

(57) ABSTRACT

Described herein are compounds and compositions for the amelioration of arthritis or joint injuries by inducing mesenchymal stem cells into chondrocytes using intra-articular administration.

### CROSS-REFERENCE

This application claims the benefit of U.S. Provisional Application Ser. No. 62/489,397 filed Apr. 24, 2017, the entirety of which is hereby incorporated by reference herein.

### BACKGROUND OF THE INVENTION

Osteoarthritis (OA) represents the most common musculoskeletal disorder. Approximately 40 million Americans are currently affected and this number is predicted to increase to 60 million within the next twenty years as a result of the aging population and an increase in life expectancy, making it the fourth leading cause of disability. OA is characterized by a degenerative breakdown of the joint including both the articular cartilage (containing the cells and matrix which produce lubrication and cushioning for the joint) and the subchondral bone underlying the articular cartilage. Current OA therapies include pain relief with oral NSAIDs or selective cyclooxygenase 2 (COX-2) inhibitors, intra-articular (IA) injection with agents such as corticorsteroids and hyaluronan, and surgical approaches.

Mesenchymal stem cells (MSCs) are present in adult articular cartilage and upon isolation can be programmed in vitro to undergo differentiation to chondrocytes and other mesenchymal cell lineages. In part it is regulated by growth factors (TGF s, BMPs), serum conditions and cell-cell contact.

### SUMMARY OF THE INVENTION

Provided herein is a method for ameliorating arthritis or joint injury in a subject, the method comprising administering to the joint space of a knee of the subject from about 10 μg to about 1000 μg of N-(4-(2-methoxyethyl)phenyl)-2-(methylsulfonamido)benzamide, or a pharmaceutically acceptable salt, or solvate thereof.

The method for ameliorating arthritis or joint injury in a subject may comprise administering to the joint space of a knee from about 10 μg to about 400 μg of N-(4-(2-methoxyethyl)phenyl)-2-(methylsulfonamido)benzamide, or a pharmaceutically acceptable salt, or solvate thereof.

The method for ameliorating arthritis or joint injury in a subject may comprise administering to the joint space of a knee from about 50 μg to about 400 μg of N-(4-(2-methoxyethyl)phenyl)-2-(methylsulfonamido)benzamide, or a pharmaceutically acceptable salt, or solvate thereof.

Provided herein is a method for ameliorating arthritis or joint injury in a subject, the method comprising administering to the joint space of a knee of the subject no more than about 1000 μg of N-(4-(2-methoxyethyl)phenyl)-2-(methylsulfonamido)benzamide, or a pharmaceutically acceptable salt, or solvate thereof.

The method for ameliorating arthritis or joint injury in a subject may comprise administering to the joint space of a knee no more than about 400 μg of N-(4-(2-methoxyethyl)phenyl)-2-(methylsulfonamido)benzamide, or a pharmaceutically acceptable salt, or solvate thereof.

Provided herein is a method for inducing differentiation of mesenchymal stem cells into chondrocytes in a subject, the method comprising administering to the joint space of a knee of the subject from about 10 μg to about 1000 μg of N-(4-(2-methoxyethyl)phenyl)-2-(methylsulfonamido)benzamide, or a pharmaceutically acceptable salt, or solvate thereof.

The method for inducing differentiation of mesenchymal stem cells into chondrocytes in a subject may comprise administering to the joint space of a knee from about 10 μg to about 400 μg of N-(4-(2-methoxyethyl)phenyl)-2-(methylsulfonamido)benzamide, or a pharmaceutically acceptable salt, or solvate thereof.

The method for inducing differentiation of mesenchymal stem cells into chondrocytes in a subject may comprise administering to the joint space of a knee from about 50 μg to about 400 μg of N-(4-(2-methoxyethyl)phenyl)-2-(methylsulfonamido)benzamide, or a pharmaceutically acceptable salt, or solvate thereof.

Disclosed herein is a method for inducing differentiation of mesenchymal stem cells into chondrocytes in a subject, the method comprising administering to the joint space of a knee of the subject not more than about 1000 μg of N-(4-(2-methoxyethyl)phenyl)-2-(methylsulfonamido)benzamide, or a pharmaceutically acceptable salt, or solvate thereof.

The method for inducing differentiation of mesenchymal stem cells into chondrocytes in a subject may comprise administering to the joint space of a knee no more than about 400 μg of N-(4-(2-methoxyethyl)phenyl)-2-(methylsulfonamido)benzamide, or a pharmaceutically acceptable salt, or solvate thereof.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject annually.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every eleven months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every ten months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every nine months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every eight months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every seven months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every six months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every five months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every four months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every three months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject every two months.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered to the subject monthly or weekly.

In the method for ameliorating arthritis or joint injury or for inducing differentiation of mesenchymal stem cells into chondrocytes in a subject, about 25 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, about 50 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, about 100 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, about 150 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, about 200 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, about 250 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, about 300 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, about 350 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, about 400 μg of N-(4-(2-methoxyethyl)phenyl)-2-(methylsulfonamido)benzamide, or a pharmaceutically acceptable salt, or solvate thereof may be administered.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered in a volume of from about 1 mL to about 5 mL.

In the method for ameliorating arthritis or joint injury or for inducing differentiation of mesenchymal stem cells into chondrocytes in a subject, N-(4-(2-methoxyethyl)phenyl)-2-(methylsulfonamido)benzamide, or a pharmaceutically acceptable salt, or solvate thereof may be administered in a volume about or no more than about 5 mL.

### BRIEF DESCRIPTION OF THE DRAWINGS

FIG. 1 shows shows the substantial cartilage degeneration width following treatment with Compound A at 10 μM.

FIG. 2 shows the combined cartilage degeneration widths following treatment with compound A once every two weeks at 10 μM.

FIG. 3 shows the total joint scores without the femur of animals treated with compound A as compared to vehicle treated animals.

FIG. 4 shows the schematic of histological analysis of osteoarthritic lesions in the tibial plateau and femur of the dog.

FIG. 5 shows the cartilage degeneration width following treatment with compound A.

FIG. 6 shows the depth of cartilage lesions in the femur following treatment with compound A.

FIG. 7 shows the levels of bone sclerosis following treatment with compound A.

FIG. 8 shows the circulating levels of collagen formation marker PIINP following treatment with compound A.

FIG. 9 shows the in vitro compound A binding to FLNA.

FIG. 10 shows the induction of CBFβ nuclear localization through compound A.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Osteoarthritis (OA) is characterized by progressive breakdown of articular cartilage, and ultimately leads to functional failure of synovial joints [Reginster, J. Y. and N. G. Khaltaev, Introduction and WHO perspective on the global burden of musculoskeletal conditions. Rheumatology (Oxford), 2002. 41 Supp 1: p. 1-2]. OA is mediated by several pathogenic mechanisms including enzymatic degradation of extracellular matrix, deficient new matrix formation, cell death, and abnormal activation and hypertrophic differentiation of cartilage cells [Goldring, M. B. and S. R. Goldring, Articular cartilage and subchondral bone in the pathogenesis of osteoarthritis. Ann N Y Acad Sci, 2010. 1192(1): p. 230-7]. The only current therapeutic options for OA are pain management and surgical intervention [Hunter, D. J., Pharmacologic therapy for osteoarthritis—the era of disease modification. Nat Rev Rheumatol, 2011. 7(1): p. 13-22].

Mesenchymal stem cells (MSCs), residing in bone marrow and most adult tissues, are capable of self-renewal and differentiation into a variety of cell lineages including chondrocytes, osteoblasts and adipocytes [Pittenger, M. F., et al., Multilineage potential of adult human mesenchymal stem cells. Science, 1999. 284(5411): p. 143-7]. Recent studies found that adult articular cartilage contains MSCs (approximately 3% of the cells) that are capable of multi-lineage differentiation. In OA cartilage, the number of these cells approximately doubles. These resident stem cells still retain the capability to differentiate into chondrocytes and thus the capacity to repair the damaged cartilage [Grogan, S. P., et al., Mesenchymal progenitor cell markers in human articular cartilage: normal distribution and changes in osteoarthritis. Arthritis Res Ther, 2009. 11(3): p. R85; Koelling, S., et al., Migratory chondrogenic progenitor cells from repair tissue during the later stages of human osteoarthritis. Cell Stem Cell, 2009. 4(4): p. 324-35].

The present invention is based, in part, on the discovery that the compounds of the present invention stimulate chondrocyte differentiation in mesenchymal stem cells. Accordingly, the present invention provides for methods of induction of mesenchymal stem cell differentiation into chondrocytes. Further, the present invention provides for administration of compounds and compositions of the present invention to prevent or ameliorate arthritis or joint injury by administrating the compound or composition into a joint, the vertebrae, vertebral disc or systemically. In particular, the compounds of the present disclosure are administered intra-articularly into the knee at a dosage of about 10 μg to about 1000 μg. The compounds may be administered as a single dose or as a course of up to four doses. Dosing may be repeated, for example, every week, two weeks, monthly, or every 3-12 months. As a non-limiting example, dosing is weekly for no more than five weeks. As used herein, administration to the knee or the joint of the knee refers to administration to one knee. However, both knees may be administered with the compounds herein. For example, each knee is dosed with about 10 μg to about 1000 μg of a compound provided herein.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “patient”, “subject” or “individual” are used interchangeably. As used herein, they refer to individuals suffering from a disorder, and the like, encompasses mammals and non-mammals. None of the terms require that the individual be under the care and/or supervision of a medical professional. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the individual is a mammal. In preferred embodiments, the individual is a human.

The terms “treat,” “treating” or “treatment,” and other grammatical equivalents as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In preferred embodiments, the compounds and compositions described herein are administered orally.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising N-(4-(2-methoxyethyl)phenyl)-2-(methylsulfonamido)benzamide required to provide a clinically significant decrease in a disease. An appropriate “effective” amount may differ from one individual to another. An appropriate “effective” amount in any individual case may be determined using techniques, such as a dose escalation study.

The term “acceptable” as used herein, with respect to a formulation, composition or ingredient, means having no persistent detrimental effect on the general health of the individual being treated.

The term “pharmaceutically acceptable” as used herein, refers to a material, such as a carrier or diluent, which does not abrogate the biological activity or properties of N-(4-(2-methoxyethyl)phenyl)-2-(methylsulfonamido)benzamide, and is relatively nontoxic, i.e., the material may be administered to an individual without causing undesirable biological effects or interacting in a deleterious manner with any of the components of the composition in which it is contained.

The term “pharmaceutically acceptable salt” as used herein, refers to salts that retain the biological effectiveness of the free acids and bases of N-(4-(2-methoxyethyl)phenyl)-2-(methylsulfonamido)benzamide and that are not biologically or otherwise undesirable. N-(4-(2-methoxyethyl)phenyl)-2-(methylsulfonamido)benzamide may react with inorganic or organic bases, and inorganic and organic acids, to form a pharmaceutically acceptable salt. These salts can be prepared in situ during the final isolation and purification, or by separately reacting the purified compound in its free base form with a suitable organic or inorganic acid, and isolating the salt thus formed.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, excipients and the like.

The term “carrier” as used herein, refers to relatively nontoxic chemical compounds or agents that facilitate the incorporation of a compound into cells or tissues.

The terms “pharmaceutical combination”, “administering an additional therapy”, “administering an additional therapeutic agent” and the like, as used herein, refer to a pharmaceutical therapy resulting from the mixing or combining of more than one active ingredient and includes both fixed and non-fixed combinations of the active ingredients. The term “fixed combination” means that N-(4-(2-methoxyethyl)phenyl)-2-(methylsulfonamido)benzamide, and at least one co-agent, are both administered to an individual simultaneously in the form of a single entity or dosage. The term “non-fixed combination” means that N-(4-(2-methoxyethyl)phenyl)-2-(methylsulfonamido)benzamide, and at least one co-agent, are administered to an individual as separate entities either simultaneously, concurrently or sequentially with variable intervening time limits, wherein such administration provides effective levels of the two or more compounds in the body of the individual. These also apply to cocktail therapies, e.g. the administration of three or more active ingredients.

The terms “co-administration”, “administered in combination with” and their grammatical equivalents or the like, as used herein, are meant to encompass administration of the selected therapeutic agents to a single individual, and are intended to include treatment regimens in which the agents are administered by the same or different route of administration or at the same or different times. In some embodiments N-(4-(2-methoxyethyl)phenyl)-2-(methylsulfonamido)benzamide will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, In some embodiments, N-(4-(2-methoxyethyl)phenyl)-2-(methylsulfonamido)benzamide and the other agent(s) are administered in a single composition. In some embodiments, N-(4-(2-methoxyethyl)phenyl)-2-(methylsulfonamido)benzamide and the other agent(s) are admixed in the composition.

“Western Ontario and McMaster Universities Arthritis Index” or “WOMAC” refers to a widely used, proprietary set of standardized questionnaires used by health professionals to evaluate the condition of patients with osteoarthritis of the knee and hip, including pain, stiffness, and physical functioning of the joints. The WOMAC has also been used to assess back pain, rheumatoid arthritis, juvenile rheumatoid arthritis, systemic lupus erythematosus, and fibromyalgia. It can be self-administered and was developed at Western Ontario and McMaster Universities in 1982. The WOMAC measures five items for pain (score range 0-20), two for stiffness (score range 0-8), and 17 for functional limitation (score range 0-68). Physical functioning questions cover everyday activities such as stair use, standing up from a sitting or lying position, standing, bending, walking, getting in and out of a car, shopping, putting on or taking off socks, lying in bed, getting in or out of a bath, sitting, and heavy and light household duties.

### Compound A

Provided herein is N-(4-(2-methoxyethyl)phenyl)-2-(methylsulfonamido)benzamide, or a pharmaceutically acceptable salt or solvate thereof:

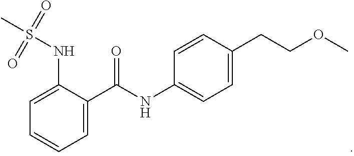

### Further Forms of Compound A

### Isomers

In some embodiments, compound A described herein exists as geometric isomers. In some embodiments, compound A described herein possesses one double bond. Compound A described herein include all cis, trans, syn, anti, entgegen (E), and zusammen (Z) isomers as well as the corresponding mixtures thereof

### Labeled Compounds

In some embodiments, compound A described herein exists in its isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compound A as pharmaceutical compositions. Thus, In some embodiments, compound A disclosed herein includes isotopically-labeled compound A, which is identical to compound A, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compound A described herein which contain the aforementioned isotopes and/or other isotopes of other atoms are within the scope of this disclosure. Certain isotopically-labeled compound A, for example those into which radioactive isotopes such as ³H and ¹⁴C are incorporated, are useful in drug and/or substrate tissue distribution assays. Tritiated, i. e., ³H and carbon-14, i. e., u isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound A, or pharmaceutically acceptable salt or solvate thereof is prepared by any suitable method.

In some embodiments, compound A described herein is labeled by other means, including, but not limited to, the use of chromophores or fluorescent moieties, bioluminescent labels, or chemiluminescent labels.

### Pharmaceutically Acceptable Salts

In some embodiments, compound A described herein exists as a pharmaceutically acceptable salt. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

Examples of pharmaceutically acceptable salts include those salts prepared by reaction of compound A with a mineral, organic acid or inorganic base, such salts including, acetate, acrylate, adipate, alginate, aspartate, benzoate, benzenesulfonate, bisulfate, bisulfate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

Further, compound A described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds of the invention and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, compound A exists as a solvate. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of compound A can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of compound A and methods provided herein.

### Tautomers

A “tautomer” as used herein refers to a proton shift from one atom of a molecule to another atom of the same molecule. Compound A presented herein may exist as a tautomer. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of compound A disclosed herein are contemplated. The exact ratio of the tautomers depends on several factors, including temperature, solvent, and pH. In some cases, Compound A may exist as:

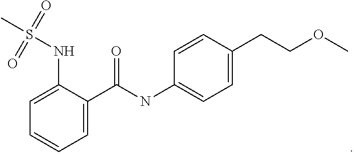

### Methods

Provided herein is a method of treating arthritis in a mammal, the method including administering about 10 μg to about 1000 μg of compound A, or a pharmaceutically acceptable salt or solvate thereof, via intra-articular injection to a joint of the mammal. For example, compound A, or a pharmaceutically acceptable salt or solvate thereof, is injected into an articulation. For example, compound A, or a pharmaceutically acceptable salt or solvate thereof, is injected into the knee. In some cases, compound A is not systemically absorbed about 1 hr, about 2 hr, about 3 hr, about 4 hr, about 5 hr, about 6 hr, about 7 hr, about 8 hr, about 9 hr, or about 10 hr after administration. In some cases, from about 10 μg to about 800 μg, from about 10 μg to about 600 μg, from about 10 μg to about 400 μg, from about 10 μg to about 200 μg, from about 10 μg to about 100 μg of compound A, or a pharmaceutically acceptable salt or solvate thereof, is administered. Compound A, or a pharmaceutically acceptable salt or solvate thereof, may be administered as a single dose or as a course of up to four doses. The dosing may be repeated, for example, weekly, bi-weekly, monthly, or every 3-12 months. As a non-limiting example, dosing is repeated every 3, 4, 5, 6, 7, 8, 9, 10, 11 or 12 months. As another non-limiting example, dosing is weekly for no more than five weeks. As another non-limiting example, dosing is biweekly.

Provided herein is a method of treating osteoarthritis in a mammal, the method including administering about 10 μg to about 1000 μg of compound A, or a pharmaceutically acceptable salt or solvate thereof, via intra-articular injection to a joint of the mammal. For example, compound A, or a pharmaceutically acceptable salt or solvate thereof, is injected into an articulation. For example, compound A, or a pharmaceutically acceptable salt or solvate thereof, is injected into the knee. In some cases, compound A is not systemically absorbed about 1 hr, about 2 hr, about 3 hr, about 4 hr, about 5 hr, about 6 hr, about 7 hr, about 8 hr, about 9 hr, or about 10 hr after administration. In some cases, from about 10 μg to about 800 μg, from about 10 μg to about 600 μg, from about 10 μg to about 400 μg, from about 10 μg to about 200 μg, from about 10 μg to about 100 μg of compound A, or a pharmaceutically acceptable salt or solvate thereof, is administered. Compound A, or a pharmaceutically acceptable salt or solvate thereof, may be administered as a single dose or as a course of up to four doses. The dosing may be repeated, for example, weekly, bi-weekly, monthly, or every 3-12 months. As a non-limiting example, dosing is repeated every 3, 4, 5, 6, 7, 8, 9, 10, 11 or 12 months. As another non-limiting example, dosing is weekly for no more than five weeks. As another non-limiting example, dosing is biweekly.

Provided herein is a method of ameliorating arthritis or joint injury in a mammal, the method including administering about 10 μg to about 1000 μg of compound A, or a pharmaceutically acceptable salt or solvate thereof, via intra-articular injection to a joint of the mammal. For example, compound A, or a pharmaceutically acceptable salt or solvate thereof, is injected into an articulation. For example, compound A, or a pharmaceutically acceptable salt or solvate thereof, is injected into the knee. In some cases, compound A is not systemically absorbed about 1 hr, about 2 hr, about 3 hr, about 4 hr, about 5 hr, about 6 hr, about 7 hr, about 8 hr, about 9 hr, or about 10 hr after administration. In some cases, from about 10 μg to about 800 μg, from about 10 μg to about 600 μg, from about 10 μg to about 400 μg, from about 10 μg to about 200 μg, from about 10 μg to about 100 μg of compound A, or a pharmaceutically acceptable salt or solvate thereof, is administered. Compound A, or a pharmaceutically acceptable salt or solvate thereof, may be administered as a single dose or as a course of up to four doses. The dosing may be repeated, for example, weekly, bi-weekly, monthly, or every 3-12 months. As a non-limiting example, dosing is repeated every 3, 4, 5, 6, 7, 8, 9, 10, 11 or 12 months. As another non-limiting example, dosing is weekly for no more than five weeks. As another non-limiting example, dosing is biweekly.

Provided herein is a method of inducing differentiation of mesenchymal stem cells into chondrocytes, the method including exposing mesenchymal stem cells by intra-articular injection to about 10 lag to about 1000 μg of compound A, or a pharmaceutically acceptable salt or solvate thereof, in a subject in need thereof, thereby inducing differentiation of the stem cells into chondrocytes. For example, compound A, or a pharmaceutically acceptable salt or solvate thereof, is injected into an articulation. For example, compound A, or a pharmaceutically acceptable salt or solvate thereof, is injected into the knee. In some cases, compound A is not systemically absorbed about 1 hr, about 2 hr, about 3 hr, about 4 hr, about 5 hr, about 6 hr, about 7 hr, about 8 hr, about 9 hr, or about 10 hr after administration. In some cases, from about 10 μg to about 800 μg, from about 10 μg to about 600 μg, from about 10 μg to about 400 μg, from about 10 μg to about 200 μg, from about 10 μg to about 100 μg of compound A, or a pharmaceutically acceptable salt or solvate thereof, is administered. Compound A, or a pharmaceutically acceptable salt or solvate thereof, may be administered as a single dose or as a course of up to four doses. The dosing may be repeated, for example, weekly, bi-weekly, monthly, or every 3-12 months. As a non-limiting example, dosing is repeated every 3, 4, 5, 6, 7, 8, 9, 10, 11 or 12 months. As another non-limiting example, dosing is weekly for no more than five weeks. As another non-limiting example, dosing is biweekly.

In some embodiments, the mammal does not have, but is at increased risk for, arthritis or joint injury. It is contemplated that the compounds, compositions, and methods of the present invention may be used to ameliorate any type of arthritis or joint injury. It is further contemplated that the compounds, compositions, and methods of the present invention may be used to ameliorate various cartilagenous disorders. In some embodiments, the compounds and compositions of the present invention are administered to prevent arthritis or joint injury, for example where there is a genetic or family history of arthritis or joint injury or prior or during joint surgery or other circumstances where there is an increased risk of arthritis or joint injury. Exemplary conditions or disorders to be treated or prevented with the compounds, compositions, and methods of the invention, include, but are not limited to systemic rheumatoid arthritis, juvenile chronic arthritis, osteoarthritis, degenerative disc disease, spondyloarthropathies, and systemic sclerosis (scleroderma). In some embodiments of the invention, the compounds, compositions, and methods of the present invention may be used to treat osteoarthritis. In some embodiments, the arthritis can be osteoarthritis, trauma arthritis, degenerative disc disease, dupuytren disease, or tendon disease.

In some embodiments, the compounds, compositions, and methods of the present invention provide a method for stimulating chondrocyte proliferation and cartilage production in cartilagenous tissues that have been damaged due to traumatic injury or chondropathy. Traumatic injury can include, but is not limited to, blunt trauma to the joint, or damage to ligaments such as tearing the anterior cruciate ligament, medial collateral ligament, or a meniscal tear. Examples of tissues that exhibit articulated surfaces, and thus are particularly susceptible to treatment include, but are not limited to, spine, shoulder, elbow, wrist, joints of the fingers, hip, knee, ankle, and the joints of the feet. Examples of diseases that may benefit from treatment include osteoarthritis, rheumatoid arthritis, other autoimmune diseases, or osteochondritis dessicans. In addition, cartilage malformation is often seen in forms of dwarfism in humans suggesting that the compounds, compositions, and methods would be useful in these patients.

It is contemplated that the compounds, compositions, and methods of the present invention may be used to treat a mammal. As used herein a “mammal” refers to any mammal classified as a mammal, including humans, domestic and farm animals, and zoo, sports or pet animals, such as cattle (e.g. cows), horses, dogs, sheep, pigs, rabbits, goats, cats, etc. In some embodiments, the mammal can be a human, a dog, a cat, or a horse. In some embodiments of the invention, the mammal is a human. In some embodiments, the mammal is a dog, a cat, or a horse. In some embodiments, the mammal is cattle, sheep, pig, goat, or rabbit. In some embodiments, the mammal is a domesticated animal or livestock. In further embodiments, the domesticated animal or livestock is a dog, cat, or horse. In some embodiments, the mammal is a companion animal. As used herein, “companion animal” refers to dog, cat, rodent, and rabbit. In some embodiments, the mammal is a companion animal or livestock. In some embodiments, the mammal is livestock.

The compounds of the present invention are also useful for inducing differentiation of mesenchymal stem cells (MSCs) into chondrocytes. In some embodiments, the present invention provides a method of inducing differentiation of mesenchymal stem cells into chondrocytes, the method including exposing mesenchymal stem cells to a sufficient amount of a compound of the present invention, thereby inducing differentiation of the stem cells into chondrocytes.

MSCs are multipotent stem cells that can differentiate into several different types of cells including, but not limited to, osteoblasts, chondrocytes and adipocytes. Differentiation is the process by which a specialized cell type is formed from a less specialized cell type, for example, a chondrocyte from a MSC. In some embodiments, the method is performed in vitro. In some embodiments, the method is performed in vivo in a mammal and the stem cells are present in the mammal. In certain embodiments, the mammal is a human, a dog, a cat, or a horse. In certain embodiments, the mammal is a human. In certain embodiments, the mammal is a dog, a cat, or a horse.

The mammal may be diagnosed or identified as having moderate to severe symptomatic osteoarthritis. For example, the mammal may be diagnosed or identified as having moderate to severe symptomatic knee osteoarthritis. In some embodiments, the mammal has grade 1 (or KL-1) osteoarthritis, as determined by the Kellgren-Lawrence system. In some embodiments, the mammal has grade 2 (or KL-2) osteoarthritis, as determined by the Kellgren-Lawrence system. In some embodiments, the mammal has grade 3 (or KL-3) osteoarthritis, as determined by the Kellgren-Lawrence system. In some embodiments, the mammal has grade 4 (or KL-4) osteoarthritis, as determined by the Kellgren-Lawrence system. In some embodiments, a mammal is administered compound A as a preventative measure, for example, a mammal with grade 1 osteoarthritis.

In some embodiments, the mammal has unilateral osteoarthritis of the knee. In some embodiments, the mammal has bilateral osteoarthritis of the knees.

In some embodiments, the mammal is overweight or obese. In some embodiments, the mammal has a body mass index (BMI) of between about 25 and about 30, for example, a BMI of 25, 26, 27, 28, or 29. In some embodiments, the mammal has a BMI of 30 or greater, such as 30, 31, 32, 33, 34, 35, 40, or greater than 40.

One method of monitoring the progression and/or treatment of osteoarthritis involves measuring the joint space. As cartilage deteriorates or wears away, narrowing of the joint space of the affected joint can be observed (joint space narrowing). Given the difficulty in measuring cartilage, joint space width (JSW) measurements are often considered a surrogate for articular cartilage thickness as such measurements involve determining the distance between two bones (e.g., using X-ray techniques). Without being bound by any theory, an increase in the JSW is an indicator of cartilage growth. Methods of measurement of JSW can be completed following radiographic imaging of the affected joint. Measurements can be either manual using calipers or a simple graduated ruler and a micrometric eyepiece or semiautomated using computer software. In some embodiments, JSW measurements can involve radiographic images (e.g., X-ray) taken of the knee. For example, one or more of metatarsophalangeal, fixed flexion, semiflexed anteroposterior (AP) and Lyon-Schuss radiographs can be used to obtain the measurement. In some embodiments, the subject is imaged while standing. For example, standing, fixed-flexion (Synaflexer), posterior-anterior (PA) radiographs.

The methods provided herein may result in an increase in the joint space width in the joint surrounding the point of injection in a mammal of compound A. The methods provided herein may result in an increase in the joint space width in the joint surrounding the point of injection in a mammal of about 5% to about 50%. For example, an increase in the joint space width in the joint surrounding the point of injection of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 50%. In some embodiments, the methods provided herein exhibit substantially no change in the joint space width at the joint surrounding the point of injection. Such a result can be indicative of an arrest of symptoms of the disease as no further loss in the joint space width is observed. The methods provided herein may result in an increase in the joint space width in the joint surrounding the point of injection in a mammal of compound A of about 0.05 mm to about 2 mm. The methods provided herein may result in an increase in the joint space width in the joint surrounding the point of injection in a mammal of about 0.05 mm; about 0.1 mm; about 0.15 mm; about 0.2 mm; about 0.25 mm; about 0.3 mm; about 0.35 mm; about 0.4 mm; about 0.45 mm; about 0.5 mm; about 0.55 mm; about 0.6 mm; about 0.65 mm; about 0.7 mm; about 0.75 mm; about 0.8 mm; about 0.85 mm; about 0.9 mm; about 0.95 mm; about 1 mm; about 1.05 mm; about 1.1 mm; about 1.15 mm; about 1.2 mm; about 1.25 mm; about 1.3 mm; about 1.35 mm; about 1.4 mm; about 1.45 mm; about 1.5 mm; about 1.55 mm; about 1.6 mm; about 1.65 mm; about 1.7 mm; about 1.75 mm; about 1.8 mm; about 1.85 mm; about 1.9 mm; about 1.95 mm; or about 2 mm. The methods provided herein may result in an increase in the joint space width in the joint surrounding the point of injection in a mammal one week after administration, or two weeks after administration, or after three weeks after administration, or after four weeks after administration, or after five weeks after administration, or after six weeks after administration, or after seven weeks after administration, or after eight weeks after administration, or after nine weeks after administration, or after 10 weeks after administration, or after 11 weeks after administration, or after twelve weeks after administration, or or after 24 weeks after administration.

The methods provided herein may result in an increase in the cartilage thickness in the joint surrounding the point of injection in a mammal of compound A. The methods provided herein may result in an increase in the cartilage thickness in the joint surrounding the point of injection in a mammal of about 5% to about 50%. For example, an increase in the cartilage thickness in the joint surrounding the point of injection of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 3′7%, about 38%, about 39%, about 40%, about 41%, about 42%, about 43%, about 44%, about 45%, about 46%, about 4′7%, about 48%, or about 50%. In some embodiments, the methods provided herein exhibit substantially no change in the cartilage thickness at the joint surrounding the point of injection. Such a result can be indicative of an arrest of symptoms of the disease as no further loss in the cartilage thickness is observed. The methods provided herein may result in an increase in the cartilage thickness in the joint surrounding the point of injection in a mammal compound A of about 0.05 mm to about 2 mm. The methods provided herein may result in an increase in the cartilage thickness in the joint surrounding the point of injection in a mammal of about 0.05 mm; about 0.1 mm; about 0.15 mm; about 0.2 mm; about 0.25 mm; about 0.3 mm; about 0.35 mm; about 0.4 mm; about 0.45 mm; about 0.5 mm; about 0.55 mm; about 0.6 mm; about 0.65 mm; about 0.7 mm; about 0.75 mm; about 0.8 mm; about 0.85 mm; about 0.9 mm; about 0.95 mm; about 1 mm; about 1.05 mm; about 1.1 mm; about 1.15 mm; about 1.2 mm; about 1.25 mm; about 1.3 mm; about 1.35 mm; about 1.4 mm; about 1.45 mm; about 1.5 mm; about 1.55 mm; about 1.6 mm; about 1.65 mm; about 1.7 mm; about 1.75 mm; about 1.8 mm; about 1.85 mm; about 1.9 mm; about 1.95 mm; or about 2 mm. The methods provided may herein result in an increase in the cartilage thickness in the joint surrounding the point of injection in a mammal one week after administration, or two weeks after administration, or after three weeks after administration, or after four weeks after administration, or after five weeks after administration, or after six weeks after administration, or after seven weeks after administration, or after eight weeks after administration, or after nine weeks after administration, or after 10 weeks after administration, or after 11 weeks after administration, or after twelve weeks after administration, or or after 24 weeks after administration.

The methods provided herein may result in a decrease in WOMAC total score in a subject. The methods provided herein may result in a decrease in WOMAC total score in the subject from baseline. For example, a decrease in WOMAC total score in the subject of at least 15 points from baseline; a decrease in WOMAC total score of at least 20 points from baseline; or a decrease in WOMAC total score of at least 25 points from baseline. The methods provided herein may result in a decrease in WOMAC total score one week after administration, or two weeks after administration, or after three weeks after administration, or after four weeks after administration, or after five weeks after administration, or after six weeks after administration, or after seven weeks after administration, or after eight weeks after administration, or after nine weeks after administration, or after 10 weeks after administration, or after 11 weeks after administration, or after twelve weeks after administration, or or after 24 weeks after administration.

The WOMAC score can be broken down into individual pain, function, and stiffness scores.

The methods provided herein may result in a decrease in WOMAC function score in a subject. The methods provided herein may result in a decrease in WOMAC function score in the subject from baseline. For example, a decrease in WOMAC function score in the subject of at least 5 points from baseline; a decrease in WOMAC function score in the subject of at least 10 points from baseline; a decrease in WOMAC function score of at least 15 points from baseline; a decrease in WOMAC function score of at least 20 points from baseline; a decrease in WOMAC function score in the subject of at least 25 points from baseline; a decrease in WOMAC function score in the subject of at least 30 points from baseline; a decrease in WOMAC function score in the subject of at least 35 points from baseline; or a decrease in WOMAC function score in the subject of at least 40 points from baseline. The methods provided herein may result in a decrease in WOMAC function score from baseline, such as, for example, a decrease in WOMAC function score of about 10% from baseline; a decrease in WOMAC function score of about 15% from baseline; or a decrease in WOMAC function score of about 20% from baseline; a decrease in WOMAC function score of about 25% from baseline; or a decrease in WOMAC function score of about 30% from baseline; a decrease in WOMAC function score of about 35% from baseline; or a decrease in WOMAC function score of about 40% from baseline; a decrease in WOMAC function score of about 45% from baseline; or a decrease in WOMAC function score of about 50% from baseline. The methods provided herein may result in a decrease in WOMAC function score one week after administration, or two weeks after administration, or after three weeks after administration, or after four weeks after administration, or after five weeks after administration, or after six weeks after administration, or after seven weeks after administration, or after eight weeks after administration, or after nine weeks after administration, or after 10 weeks after administration, or after 11 weeks after administration, or after twelve weeks after administration, or or after 24 weeks after administration.

The methods provided herein may result in a decrease in WOMAC pain score in a subject. The methods provided herein may result in a decrease in WOMAC pain score in the subject from baseline. For example, a decrease in WOMAC pain score in the subject of at least 6 points from baseline; a decrease in WOMAC pain score in the subject of at least 8 points from baseline; a decrease in WOMAC pain score of at least 10 points from baseline; a decrease in WOMAC pain score in the subject of at least 12 points from baseline; or a decrease in WOMAC pain score in the subject of at least 14 points from baseline. The methods provided herein may result in a decrease in WOMAC pain score from baseline, such as, for example, a decrease in WOMAC pain score of about 10% from baseline; a decrease in WOMAC pain score of about 15% from baseline; or a decrease in WOMAC pain score of about 20% from baseline; a decrease in WOMAC pain score of about 25% from baseline; or a decrease in WOMAC pain score of about 30% from baseline; a decrease in WOMAC pain score of about 35% from baseline; or a decrease in WOMAC pain score of about 40% from baseline; a decrease in WOMAC pain score of about 45% from baseline; or a decrease in WOMAC pain score of about 50% from baseline. The methods provided herein may result in a decrease in WOMAC pain score one week after administration, or two weeks after administration, or after three weeks after administration, or after four weeks after administration, or after five weeks after administration, or after six weeks after administration, or after seven weeks after administration, or after eight weeks after administration, or after nine weeks after administration, or after 10 weeks after administration, or after 11 weeks after administration, or after twelve weeks after administration, or or after 24 weeks after administration.

The methods provided herein may result in a decrease in WOMAC stiffness score in a subject. The methods provided herein may result in a decrease in WOMAC stiffness score in the subject from baseline. For example, a decrease in WOMAC stiffness score in the subject of at least 2 points from baseline; a decrease in WOMAC stiffness score in the subject of at least 3 points from baseline; a decrease in WOMAC stiffness score of at least 4 points from baseline; or a decrease in WOMAC stiffness score of at least 5 points from baseline. The methods provided herein may result in a decrease in WOMAC stiffness score from baseline, such as, for example, a decrease in WOMAC stiffness score of about 10% from baseline; a decrease in WOMAC stiffness score of about 15% from baseline; or a decrease in WOMAC stiffness score of about 20% from baseline; a decrease in WOMAC stiffness score of about 25% from baseline; or a decrease in WOMAC stiffness score of about 30% from baseline; a decrease in WOMAC stiffness score of about 35% from baseline; or a decrease in WOMAC stiffness score of about 40% from baseline; a decrease in WOMAC stiffness score of about 45% from baseline; or a decrease in WOMAC stiffness score of about 50% from baseline. The methods provided herein may result in a decrease in WOMAC stiffness score one week after administration, or two weeks after administration, or after three weeks after administration, or after four weeks after administration, or after five weeks after administration, or after six weeks after administration, or after seven weeks after administration, or after eight weeks after administration, or after nine weeks after administration, or after 10 weeks after administration, or after 11 weeks after administration, or after twelve weeks after administration, or or after 24 weeks after administration.

The methods provided herein may result in a decrease in WORMS score (Whole-Organ Magnetic Resonance Imaging Score) in a subject. The methods provided herein may result in a decrease in WORMS score in the subject from baseline. For example, a decrease in WORMS score in the subject of at least 10 points from baseline; a decrease in WORMS score in the subject of at least 15 points from baseline; a decrease in WORMS score of at least 20 points from baseline; or a decrease in WORMS score of at least 25 points from baseline; or a decrease in WORMS score of at least 30 points from baseline; or a decrease in WORMS score of at least 35 points from baseline; or a decrease in WORMS score of at least 40 points from baseline; or a decrease in WORMS score of at least 45 points from baseline; or a decrease in WORMS score of at least 50 points from baseline; or a decrease in WORMS score of at least 55 points from baseline; or a decrease in WORMS score of at least 60 points from baseline; or a decrease in WORMS score of at least 65 points from baseline; or a decrease in WORMS score of at least 70 points from baseline; or a decrease in WORMS score of at least 75 points from baseline; or a decrease in WORMS score of at least 80 points from baseline; or a decrease in WORMS score of at least 85 points from baseline; or a decrease in WORMS score of at least 90 points from baseline; or a decrease in WORMS score of at least 95 points from baseline; or a decrease in WORMS score of at least 100 points from baseline. The methods provided herein may result in a decrease in WORMS score from baseline, such as, for example, a decrease in WORMS score of about 10% from baseline; a decrease in WORMS score of about 15% from baseline; or a decrease in WOMAC score of about 20% from baseline; a decrease in WORMS score of about 25% from baseline; or a decrease in WORMS score of about 30% from baseline; a decrease in WORMS score of about 35% from baseline; or a decrease in WORMS score of about 40% from baseline; a decrease WORMS score of about 45% from baseline; or a decrease in WORMS score of about 50% from baseline. The methods provided herein may result in a decrease in WORMS score one week after administration, or two weeks after administration, or after three weeks after administration, or after four weeks after administration, or after five weeks after administration, or after six weeks after administration, or after seven weeks after administration, or after eight weeks after administration, or after nine weeks after administration, or after 10 weeks after administration, or after 11 weeks after administration, or after twelve weeks after administration, or or after 24 weeks after administration.

The methods provided herein may result in an increase of the N-propeptide of type IIA collagen (PIIANP) serum level. Type II collagen is the most abundant protein of cartilage matrix and alterations in turnover of this molecule are believed to play a role in the progressive loss of cartilage in osteaoarthritis. Type II procollagen is synthesized in two splice forms, type IIA and type IIB. The N-propeptide of type IIA collagen (PIIANP) can be specifically measured and may represent a biological marker of phenotypic changes of chondrocytes. It has been shown that serum levels of type IIA procollagen amino terminal propeptide (PIIANP) are decreased in patients with knee osteoarthritis. Serum levels of PIIANP may be used as a potential biomarker for type II collagen synthesis.

The methods provided herein may result in an increase in N-propeptide of type IIA collagen (PIIANP) serum level, such as, for example, an increase in N-propeptide of type IIA collagen (PIIANP) serum level between about 5% and about 50%, or an increase in N-propeptide of type IIA collagen (PIIANP) serum level of about 5% from baseline; or an increase in N-propeptide of type IIA collagen (PIIANP) serum level of about 10% from baseline; or an increase in N-propeptide of type IIA collagen (PIIANP) serum level of about 15% from baseline; or an increase in N-propeptide of type IIA collagen (PIIANP) serum level of about 20% from baseline; or an increase in N-propeptide of type IIA collagen (PIIANP) serum level of about 25% from baseline; or an increase in N-propeptide of type IIA collagen (PIIANP) serum level of about 30% from baseline; or an increase in N-propeptide of type IIA collagen (PIIANP) serum level of about 35% from baseline; or an increase in N-propeptide of type IIA collagen (PIIANP) serum level of about 40% from baseline; or an increase in N-propeptide of type IIA collagen (PIIANP) serum level of about 45% from baseline; or an increase in N-propeptide of type IIA collagen (PIIANP) serum level of about 50% from baseline. The methods provided herein may result in an increase in N-propeptide of type IIA collagen (PIIANP) serum level one week after administration, or two weeks after administration, or after three weeks after administration, or after four weeks after administration, or after five weeks after administration, or after six weeks after administration, or after seven weeks after administration, or after eight weeks after administration, or after nine weeks after administration, or after 10 weeks after administration, or after 11 weeks after administration, or after twelve weeks after administration, or or after 24 weeks after administration.

### Preparation of Compound A

Described herein is compound A, or a pharmaceutically acceptable salt or solvate thereof, for inducing differentiation of mesenchymal stem cells into chondrocytes and for ameliorating arthritis or joint injury in a mammal, and processes for the preparation of this compound. Pharmaceutical compositions comprising compound A or a pharmaceutically acceptable salt or solvate of such compound, and a pharmaceutically acceptable excipient are also provided.

Compound A described herein may be synthesized using standard synthetic reactions known to those of skill in the art or using methods known in the art. The reactions can be employed in a linear sequence to provide compound A or they may be used to synthesize fragments which are subsequently joined by the methods known in the art.

The starting material used for the synthesis of compound A may be synthesized or can be obtained from commercial sources, such as, but not limited to, Aldrich Chemical Co. (Milwaukee, Wis.), Bachem (Torrance, Calif.), or Sigma Chemical Co. (St. Louis, Mo.). compound A, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989). (all of which are incorporated by reference in their entirety). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography and the like. Such materials may be characterized using conventional means, including physical constants and spectral data.

### Pharmaceutical Compositions/Formulations

In another aspect, provided herein are pharmaceutical compositions comprising compound A, or a pharmaceutically acceptable salt or solvate thereof, and a pharmaceutically acceptable excipient.

In some embodiments, compound A, or a pharmaceutically acceptable salt or solvate thereof, is formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Provided herein are pharmaceutical compositions that include compound A, or a pharmaceutically acceptable salt or solvate thereof, and at least one pharmaceutically acceptable inactive ingredient. In some embodiments, the compounds described herein are administered as pharmaceutical compositions in which a compound described herein is mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of compound A, or a pharmaceutically acceptable salt or solvate thereof, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human, a dog, a cat, or a horse. In some embodiments, the mammal is a human. In some embodiments, the mammal is a dog, a cat, or a horse. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical composition described herein may be in the form of a liquid for intra-articular injection. The pharmaceutical composition described herein my comprise compound A, or a pharmaceutically acceptable salt thereof in an amount between about 0.05 g to about 3 g per 1 L of intra-articular liquid formulation. The amount of compound A, or a pharmaceutically acceptable salt thereof may be about 0.05 g, about 0.06 g, about 0.07 g, about 0.08 g, about 0.09 g, about 0.1 g, about 0.2 g, about 0.3 g, about 0.4 g, about 0.5 g, about 0.6 g, about 0.7 g about 0.8 g, about 0.9 g, about 1 g, about 1.1 g, about 1.2 g, about 1.3 g, about 1.4 g, about 1.5 g, about 1.6 g, about 1.7 g about 1.8 g, about 1.9 g, about 2 g, about 2.1 g, about 2.2 g, about 2.3 g, about 2.4 g, about 2.5 g, about 2.6 g, about 2.7 g about 2.8 g, about 2.9 g, or about 3 g per per 1 L of intra-articular liquid formulation.

The pharmaceutical composition described herein may be in the form of a liquid for intra-articular injection and may further comprise a pharmaceutically acceptable carrier. The carrier may be an aqueous carrier.

The pharmaceutical composition described herein may be in the form of a liquid for intra-articular injection and may further comprise a pharmaceutically acceptable excipient. Pharmaceutically acceptable excipient may include solvents, co-solvents, surfactants, buffers, solubilizers, tonicity agents, stabilizers, preservatives, viscosity enhancers, and anti-foaming agents or any combinations thereof. Methods of preparing such pharmaceutical composition are known, or will be apparent, to those skilled in the art; for example, see Remington: The Science and Practice of Pharmacy, 22nd Edition (Pharmaceutical Press, London, U K. 2012).

The pharmaceutical composition described herein may be in the form of a liquid for intra-articular injection and may further comprise a solvent. The pharmaceutical composition described herein may be in the form of a liquid for intra-articular injection and may further comprise multiple solvents. The solvents may be selected from polyethylene glycols and alcohols. The solvents may be selected from PEG 3350 and benzyl alcohol.

The pharmaceutical composition described herein may be in the form of a liquid for intra-articular injection and may further comprise a co-solvent. The co-solvents may be selected from glycols such as polyethylene glycol (PEG 200, PEG 300, or PEG 400) or propylene glycol; alcohols such as isopropanol, propanol, or ethanol; N,N-dimethylacetamide (DMA): N-methyl-2-pyrrolidone (NMP); polyvinylpyrrolidone (PVP), dimethylsulphoxide (DMSO); and any combinations thereof.

The pharmaceutical composition described herein may be in the form of a liquid for intra-articular injection and may further comprise a surfactant. Non-limiting examples of surfactants include polysorbates such as polysorbate 20, polysorbate 40, polysorbate 60, polysorbate 80, and polysorbate 85; polyoxyethylene hydrogenated castor oils such as polyoxyethylene hydrogenated castor oil 60 and polyoxyl 35 castor oil, sorbitan fatty acid esters; sucrose fatty acid esters; polyoxyethylene polyoxypropylene glycols; polyoxyethylene fatty acid ethers; polyoxyl stearates: and other surfactants, including, but not limited to, 1,2-dimyristoyl-sn-glycero-3-(phospho-s-(1-glycerol)), 1,2-dioleoyl-sn-glycero-3-phosphocholine, 1,2-dipalmitoyl-sn-glycero-3-(phospho-rac-(1-glycerol)), 1,2-distearoyl-sn-glycero-3-(phospho-rac-(1-glycerol)), 1,2-distearoyl-sn-glycero-3-phosphocholine, deoxycholic acid, dipalmitoylphosphatidylglycerol (dl), distearoylphosphatidylcholine (dl), docusate sodium, egg phospholipids, glyceryl palmitostearate, glyceryl trioleate, hydrogenated soybean lecithin, hydrolyzed soy protein (enzymatic; 2000 mw), hydroxyethylpiperazine ethane sulfonic acid, lecithin, miripirium chloride, n-(carbonyl-methoxypolyethylene glycol 2000)-1,2-distearoyl-sn-glycero-3-phiv, oleic acid, palmitic acid, peg vegetable oil, peg-20 sorbitan isostearate, peg-40 castor oil, phospholipid, poloxamer 188, polyethylene glycol 200, polyethylene glycol 300, polyethylene glycol 3350, polyethylene glycol 400, polyethylene glycol 4000, polyethylene glycol 600, polyoxyethylene fatty acid esters, sodium cholesteryl sulfate, sodium deoxycholate, sodium n-(carbonyl-methoxypoly ethylene glycol 2000)-4,2-distearoyl-sn-glyc, sodium oleate, sorbitan monolaurate, sorbitan monopalmitate, stearic acid, tricaprylin, or mixtures thereof.

The pharmaceutical composition described herein may be in the form of a liquid for intra-articular injection and may further comprise a buffer to maintain the pH between about 5 and about 9. The pH may be adjusted with the addition of an acid such as hydrochloric acid. The pH may be maintained at a pH suitable for injection. The pH may be maintained at a pH of about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. Examples of buffer agents include, but are not limited to, acetic acid, acetic anhydride, adipic acid, alanine, albumin, alcohol, alfadex, ammonia, ammonium acetate, ammonium sulfate, anhydrous citric acid, anhydrous dextrose, anhydrous lactose, anhydrous trisodium citrate, arginine, ascorbic acid, aspartic acid, benzenesulfonic acid, benzoic acid, calcium chloride, calcium gluceptate, calcium hydroxide, calcium, caprylic acid, carbon dioxide, citric acid monohydrate, dibasic potassium phosphate, diethanolamine, disodium citrate sesquihydrate, disodium hydrogen citrate, edetate calcium disodium, edetate disodium, edetate sodium, edetic acid, ethanolamine hydrochloride, ferric chloride, gluceptate sodium, glycine hydrochloride, glycine, guanidine hydrochloride, histidine, hydrochloric acid, isoleucine, lactic acid, lactobionic acid, leucine, lysine acetate, lysine, lysine monohydrate, magnesium chloride, magnesium stearate, maleic acid, metaphosphoric acid, methanesulfonic acid, nitric acid, phosphate ion, phosphoric acid, potassium chloride, potassium hydroxide, potassium phosphate (monobasic), sodium acetate, sodium ascorbate, sodium benzoate, sodium bicarbonate, sodium bisulfate, sodium carbonate, sodium citrate, sodium hydroxide, sodium hypochlorite, sodium phosphate dihydrate, sodium phosphate, sodium phosphate dibasic dihydrate, sodium phosphate dibasic dodecahydrate, sodium phosphate dibasic, sodium phosphate dibasic (anhydrous), sodium phosphate dibasic heptahydrate, sodium phosphate monobasic (anhydrous), sodium phosphate monobasic dihydrate, sodium phosphate monobasic monohydrate, sodium phosphate monobasic, sodium sulfate (anhydrous), sodium sulfate, sodium thioglycolate, sodium thiomalate, sodium thiosulfate, succinic acid, sulfuric acid, tartaric acid, tartaric acid (dl), trifluoroacetic acid, tromantadine, and tromethamine.

The pharmaceutical composition described herein may be in the form of a liquid for intra-articular injection and may further comprise a solubilizer. Examples of solubilizers include, but are not limited to, acetyltryptophan (dl), alanine, albumin (aggregated), alcohol, alfadex intracavitary powder, ammonia, anhydrous dextrose, anhydrous lactose, anhydrous trisodium citrate, arginine, ascorbic acid, aspartic acid, benzenesulfonic acid, benzyl alcohol, benzyl benzoate, benzyl chloride, betadex sulfobutyl ether sodium, butanol (mixed isomers), caprylic acid, carboxymethylcellulose, carboxymethylcellulose sodium, castor oil, cholesterol, corn oil, cottonseed oil, creatine, creatinine, croscarmellose sodium, crospovidone, cysteine hydrochloride, cysteine, cysteine (dl), dextran 40, dextran, diacetylated monoglycerides, diethanolamine, dimethyl sulfoxide, ethanolamine hydrochloride, ethyl acetate, ethylene-vinyl acetate copolymer (15% vinyl acetate), gamma cyclodextrin, gelatin, gentisic acid ethanolamide, gentisic acid, gluconolactone, glucuronic acid, glycerin, hetastarch, human albumin microspheres, hyaluronate sodium, hydroxypropyl betadex intramuscular injection, hypromellose, isopropyl alcohol, methylcellulose, methylpyrrolidone, microcrystalline cellulose, N,N-dimethylacetamide, niacinamide, oleic acid, palmitic acid, peanut oil, peg vegetable oil, peg-20 sorbitan isostearate, peg-40 castor oil, phenylethyl alcohol, polyethylene glycol 200, polyethylene glycol 300, polyethylene glycol 3350, polyethylene glycol 400, polyethylene glycol 4000, polyethylene glycol 600, polypropylene glycol, polyvinyl alcohol, poppy seed oil, povidone k12, povidone k17, povidone, proline, propyl gallate, propylene glycol, sesame oil, soybean oil, starch, stearic acid, trimethylsilyl treated dimethiconol/trimethylsiloxysilicate crosspolymer, and yellow wax, and combinations thereof.

The pharmaceutical composition described herein may be in the form of a liquid for intra-articular injection and may further comprise a tonicity agent. Examples of tonicity agents include, but are not limited to, dextrose monohydrate, dextrose solution, dextrose, dimethyl sulfoxide, fructose, gluconolactone, glucuronic acid, glycerin, glycine hydrochloride, glycine, guanidine hydrochloride, histidine, hydrochloric acid, hypertonic sodium chloride solution, isoleucine, isopropyl alcohol, isotonic sodium chloride solution, lactic acid (dl), lactobionic acid, lactose monohydrate, lactose, leucine, lysine acetate, lysine, lysine monohydrate, magnesium chloride, magnesium stearate, maleic acid, mannitol, meglumine, methionine, methylboronic acid, polypropylene glycol, potassium chloride, potassium hydroxide, potassium phosphate (monobasic), proline, propyl gallate, propylene glycol, saccharin sodium, serine, sodium acetate, sodium ascorbate, sodium benzoate, sodium bicarbonate, sodium bisulfate, sodium carbonate, sodium chloride, sodium citrate, sodium gluconate, sodium hydroxide, sodium hypochlorite, sodium lactate, sodium phosphate dihydrate, sodium phosphate, sodium phosphate dibasic dihydrate, sodium phosphate dibasic dodecahydrate, sodium phosphate dibasic, sodium phosphate dibasic (anhydrous), sodium phosphate dibasic heptahydrate, sodium phosphate monobasic (anhydrous), sodium phosphate monobasic dihydrate, sodium phosphate monobasic monohydrate, sodium phosphate monobasic, sodium sulfate (anhydrous), sodium sulfate, sodium thioglycolate, sodium thiomalate, sodium thiosulfate, sorbitol, succinic acid, sucrose, sulfuric acid, tartaric acid, tartaric acid (dl), threonine, trehalose, trifluoroacetic acid, trisodium citrate dihydrate, tromethamine, tryptophan, tyrosine, urea, urethane, and valine and combinations thereof.

The pharmaceutical composition described herein may be in the form of a liquid for intra-articular injection and may further comprise a stabilizer. Examples of stabilizers include, but are not limited to, acetyltryptophan (dl), alanine, albumin (aggregated), alcohol, alfadex intracavitary powder, ammonia, anhydrous dextrose, anhydrous lactose, anhydrous trisodium citrate, arginine, ascorbic acid, aspartic acid, benzenesulfonic acid, benzyl alcohol, benzyl benzoate, benzyl chloride, betadex sulfobutyl ether sodium, boric acid, butanol (mixed isomers), caprylic acid, carboxymethylcellulose, carboxymethylcellulose sodium, castor oil, cholesterol, creatine, creatinine, croscarmellose sodium, crospovidone, cysteine hydrochloride, cysteine, cysteine (dl), dextran 40, dextran, ethylene-vinyl acetate copolymer (15% vinyl acetate), gelatin, gentisic acid ethanolamide, gentisic acid, hetastarch, human albumin microspheres, hyaluronate sodium, hypromellose, meglumine, methionine, methylboronic acid, methylcellulose, methylpyrrolidone, microcrystalline cellulose, miripirium chloride, N-(carbonyl-methoxypoly ethylene glycol 2000)-1,2-distearoyl-sn-glycero-3-phiv, N,N-dimethylacetamide, niacinamide, phenylalanine, polyvinyl alcohol, povidone K12, povidone K17, povidone, serine, sodium citrate, sodium gluconate, sodium lactate, starch, threonine, trehalose, tricaprylin, trimethylsilyl treated dimethiconol/trimethylsiloxysilicate crosspolymer, tri sodium citrate dihydrate, tryptophan, tyrosine, urea, and valine and combinations thereof.

The pharmaceutical composition described herein may be in the form of a liquid for intra-articular injection and may further comprise a preservative. Examples of preservatives include, but are not limited to, acetone sodium bisulfite, alpha-tocopherol, benzalkonium chloride, benzyl alcohol, benzyl benzoate, benzyl chloride, boric acid, butylated hydroxyanisole, butylated hydroxytoluene, butylparaben, chlorobutanol, chlorobutanol hemihydrate, cresol, diethyl pyrocarbonate, edetate calcium disodium, edetate disodium, edetate sodium, edetic acid, hexylresorcinol, metacresol, methylparaben, miripirium chloride, monothioglycerol, nitrogen, phenol, phenylethyl alcohol, phenylmercuric nitrate, potassium bisulfite, potassium metabi sulfite, propylparaben, sodium ascorbate, sodium benzoate, sodium bisulfate, sodium chlorate, sodium dithionite, sodium formaldehyde sulfoxylate, sodium iodide, sodium metabi sulfite, sodium sulfite, sodium tartrate, sulfur dioxide, sulfurous acid, and thimerosal and combinations thereof.

The pharmaceutical composition described herein may be in the form of a liquid for intra-articular injection and may further comprise a viscosity enhancer. Examples of viscosity enhancers include, but are not limited to, carboxymethylcellulose, carboxymethylcellulose sodium, croscarmellose sodium, crospovidone, ethylene-vinyl acetate copolymer (15% vinyl acetate), gelatin, hetastarch, human albumin microspheres, hyaluronate sodium, hypromellose, methylcellulose, methylpyrrolidone, microcrystalline cellulose, polyvinyl alcohol, povidone K12, povidone K17, povidone, starch, and trimethylsilyl treated dimethiconol/trimethylsiloxysilicate crosspolymer and combinations thereof.

The pharmaceutical composition described herein may be in the form of a liquid for intra-articular injection and may further comprise an anti-foaming agent. Examples of anti-foaming agents include, but are not limited to, dimethicone, polysiloxane, silicone, and simethicone, and combinations thereof.

### Stability

The compositions described herein are stable in various storage conditions including refrigerated, ambient and accelerated conditions. Stable as used herein refer to formulations having about 95% of the compound of compound A and about 5% or less total impurities or related substances at the end of a given storage period. Stability is assessed by HPLC or any other known testing method. The stable formulations may have about 5%, about 4%, about 3%, about 2.5%, about 2%, about 1.5%, about 1%, or about 0.5% total impurities or related substances. The stable formulations may have about 5% total impurities or related substances. The stable formulations may have about 4% total impurities or related substances. The stable formulations may have about 3% total impurities or related substances. The stable formulations may have about 2% total impurities or substances. The stable formulations may have about 1% total impurities or related substances. The stable formulations may have about 95%, about 96%, about 97%, about 98% or about 99% of compound A at the end of a given storage period.

At refrigerated and ambient conditions, the formulations described herein are stable for at least 1 month. At refrigerated and ambient conditions, the formulations described herein are stable for at least 30 days, at least 29 days, at least 28 days, at least 27 days, at least 26 days, at least 25 days, at least 24 days, at least 23 days, at least 22 days, at least 21 days, at least 20 days, at least 19 days, at least 18 days, at least 17 days, at least 16 days, at least 15 days, at least 14 days, at least 13 days, at least 12 days, at least 11 days, at least 10 days, at least 9 days, at least 8 days, at least 7 days, at least 6 days, at least 5 days, at least 4 days, at least 3 days, at least 2 days, or at least 1 day. In some instances, a refrigerated condition is at about 2° C., about 3° C., about 4° C., about 5° C., about 6° C., about 7° C. or about 8° C. In other instances, a refrigerated condition is at about 4° C.

At accelerated conditions, the formulations described herein are stable for at least 1 month. At accelerated conditions, the formulations described herein are stable for at least 30 days, at least 29 days, at least 28 days, at least 27 days, at least 26 days, at least 25 days, at least 24 days, at least 23 days, at least 22 days, at least 21 days, at least 20 days, at least 19 days, at least 18 days, at least 17 days, at least 16 days, at least 15 days, at least 14 days, at least 13 days, at least 12 days, at least 11 days, at least 10 days, at least 9 days, at least 8 days, at least 7 days, at least 6 days, at least 5 days, at least 4 days, at least 3 days, at least 2 days, or at least 1 day. Accelerated conditions include temperature and/or relative humidity (RH) that are above ambient levels (e.g. 25±5° C.; 55±10% RH). In some instances, an accelerated condition is at about 30° C., about 35° C., about 40° C., about 45° C., about 50° C., about 55° C. or about 60° C. In other instances, an accelerated condition is above 65% RH, about 70% RH, about 75% RH or about 80% RH. In further instances, an accelerated condition is about 40° C. or 60° C. at ambient humidity. In yet further instances, an accelerated condition is about 40° C. at 75±5% RH humidity. Ambient conditions include temperature and/or relative humidity (RH) that are at ambient levels (e.g. 25±5° C.; 55±10% RH). In some instances, an ambient condition is at about 20° C., about 21° C., about 22° C., about 23° C., about 24° C., about 25° C., about 26° C., about 27° C., about 28° C., about 29° C., or about 30° C. In other instances, an ambient condition is about 45% RH, about 50% RH, about 55% RH, about 60% RH or about 65% RH. Refrigerated conditions include temperature and/or relative humidity (RH) in typical refrigeration units (e.g., 5±3° C.).

### Doses

The amount of pharmaceutical composition administered comprising compound A, or a pharmaceutically acceptable salt or solvate thereof, will firstly be dependent on the mammal being treated. In the instances where pharmaceutical compositions are administered to a human individual, the daily dosage will normally be determined by the prescribing physician with the dosage generally varying according to the age, sex, diet, weight, general health and response of the individual, the severity of the individual's symptoms, the precise indication or condition being treated, the severity of the indication or condition being treated, time of administration, the disposition of the composition, rate of excretion, drug combination, and the discretion of the prescribing physician. Preferably, the pharmaceutical composition is administered by intra-articular injection into a joint, for example, the knee. In some instances, treatment may be initiated with smaller dosages which are less than the optimum dose and thereafter, the dosage may be increased by small amounts until the optimum effect under the circumstances is reached. The amount and frequency of administration of the composition herein, and if applicable other therapeutic agents and/or therapies, will be regulated according to the judgment of the attending clinician (physician) considering such factors as described above. Thus the amount of pharmaceutical composition to be administered may vary widely.

Intra-articular administration of a composition comprising compound A, or a pharmaceutically acceptable salt or solvate thereof, herein may occur in an amount of about 10 μg to about 1000 μg. A particular therapeutic dosage can include, e.g., from about 10 μg to about 50 μg, from about 10 μg to about 100 μg, from about 10 μg to about 200 μg, from about 10 μg to about 300 μg, from about 10 μg to about 400 μg, from about 10 μg to about 500 μg, from about 10 μg to about 600 μg, from about 10 μg to about 700 μg, from about 10 μg to about 800 μg, from about 10 μg to about 900 μg, from about 10 μg to about 1000 μg, from about 50 μg to about 100 μg, from about 50 μg to about 200 μg, from about 50 μg to about 300 μg, from about 50 μg to about 400 μg, from about 50 μg to about 500 μg, from about 50 μg to about 600 μg, from about 50 μg to about 700 μg, from about 50 μg to about 800 μg, from about 50 μg to about 900 μg, from about 50 μg to about 1000 μg, from about 100 μg to about 200 μg, from about 100 μg to about 300 μg, from about 100 μg to about 400 μg, from about 100 μg to about 500 μg, from about 100 μg to about 600 μg, from about 100 μg to about 700 μg, from about 100 μg to about 800 μg, from about 100 μg to about 900 μg, from about 100 μg to about 1000 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, from about 500 μg to about 550 μg, from about 550 μg to about 600 μg, about 600 μg to about 650 μg, from about 650 μg to about 700 μg, about 700 μg to about 750 μg, from about 750 μg to about 800 μg, about 800 μg to about 950 lag, from about 950 μg to about 1000 μg, or any range there between. In some cases, from about 10 μg to about 100 μg is administered per joint. In some cases, from about 50 μg to about 200 μg is administered per joint. In some cases, from about 200 μg to about 800 μg is administered per joint. In some cases, the therapeutic dose is less than about 1000 μg, less than about 900 μg, less than about 800 μg, less than about 700 μg, less than about 600 μg, less than about 500 μg, less than about 400 μg, less than about 300 μg, less than about 200 μg, or less than about 100 μg. In some cases, the therapeutic dose does not exceed about 1000 μg, does not exceed about 900 μg, does not exceed about 800 μg, does not exceed about 700 μg, does not exceed about 600 μg, does not exceed about 500 μg, does not exceed about 400 μg, does not exceed about 300 μg, does not exceed about 200 μg, or does not exceed about 100 μg per joint. A particular therapeutic dosage per joint can include, e.g., from about 10 μg to about 50 μg, from about 10 μg to about 100 μg, from about 10 μg to about 200 μg, from about 10 μg to about 300 μg, from about 10 μg to about 400 μg, from about 10 μg to about 500 μg, from about 10 μg to about 600 μg, from about 10 μg to about 700 μg, from about 10 μg to about 800 μg, from about 10 μg to about 900 μg, from about 10 μg to about 1000 μg, from about 50 μg to about 100 μg, from about 50 μg to about 200 μg, from about 50 μg to about 300 μg, from about 50 μg to about 400 μg, from about 50 μg to about 500 μg, from about 50 μg to about 600 μg, from about 50 μg to about 700 μg, from about 50 μg to about 800 μg, from about 50 μg to about 900 μg, from about 50 μg to about 1000 μg, from about 100 μg to about 200 μg, from about 100 μg to about 300 μg, from about 100 μg to about 400 μg, from about 100 μg to about 500 μg, from about 100 μg to about 600 μg, from about 100 μg to about 700 μg, from about 100 μg to about 800 μg, from about 100 μg to about 900 μg, from about 100 μg to about 1000 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, from about 500 μg to about 550 μg, from about 550 μg to about 600 μg, about 600 μg to about 650 μg, from about 650 μg to about 700 μg, about 700 μg to about 750 μg, from about 750 μg to about 800 μg, about 800 μg to about 950 lag, from about 950 μg to about 1000 μg, or any range there between. In some cases, from about 10 μg to about 100 μg is administered per mammal. In some cases, from about 50 μg to about 200 μg is administered per mammal. In some cases, from about 200 μg to about 800 μg is administered per mammal. In some cases, the therapeutic dose is less than about 1000 ng, less than about 900 ng, less than about 800 ns, less than about 700 μg, less than about 600 μg, less than about 500 μg, less than about 400 μg, less than about 300 μg, less than about 200 μg, or less than about 100 μg. In some cases, the therapeutic dose does not exceed about 1000 μg, does not exceed about 900 μg, does not exceed about 800 μg, does not exceed about 700 μg, does not exceed about 600 μg, does not exceed about 500 μg, does not exceed about 400 μg, does not exceed about 300 μg, does not exceed about 200 μg, or does not exceed about 100 μg per mammal. A particular therapeutic dosage per mammal can include, e.g., from about 10 μg to about 50 μg, from about 10 μg to about 100 μg, from about 10 μg to about 200 μg, from about 10 μg to about 300 μg, from about 10 μg to about 400 μg, from about 10 μg to about 500 μg, from about 10 μg to about 600 μg, from about 10 μg to about 700 μg, from about 10 μg to about 800 μg, from about 10 μg to about 900 μg, from about 10 μg to about 1000 μg, from about 50 μg to about 100 μg, from about 50 μg to about 200 μg, from about 50 μg to about 300 μg, from about 50 μg to about 400 μg, from about 50 μg to about 500 μg, from about 50 μg to about 600 μg, from about 50 μg to about 700 μg, from about 50 μg to about 800 μg, from about 50 μg to about 900 μg, from about 50 μg to about 1000 μg, from about 100 μg to about 200 μg, from about 100 μg to about 300 μg, from about 100 μg to about 400 μg, from about 100 μg to about 500 μg, from about 100 μg to about 600 μg, from about 100 μg to about 700 μg, from about 100 μg to about 800 μg, from about 100 μg to about 900 μg, from about 100 μg to about 1000 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, from about 500 μg to about 550 μg, from about 550 μg to about 600 μg, about 600 μg to about 650 μg, from about 650 μg to about 700 μg, about 700 μg to about 750 μg, from about 750 μg to about 800 μg, about 800 μg to about 950 lag, from about 950 μg to about 1000 μg, or any range there between.

Intra-articular administration of a composition comprising compound A, or a pharmaceutically acceptable salt or solvate thereof, may occur in an amount of about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg. In some cases, the therapeutic dose does not exceed about 1000 lag, does not exceed about 900 μg, does not exceed about 800 μg, does not exceed about 700 μg, does not exceed about 600 μg, does not exceed about 500 μg, does not exceed about 400 μg, does not exceed about 300 μg, does not exceed about 200 μg, or does not exceed about 100 μg. In some cases, the therapeutic dose is administered in 1, 2, 3, 4, 5, 6, 7, 8, 9, or 10 injections. For example, the therapeutic dose is administered once. As another example, the therapeutic dose is administered 2, 3, or 4 times. In some instances, dosage levels below the lower limit of the aforesaid range may be more than adequate, while in other cases still larger doses may be employed without causing any harmful side effect, e.g. by dividing such larger doses into several small doses. The therapeutic dosing of the composition described in the examples may also be used for treatment.

In some embodiments, a composition comprising compound A, or a pharmaceutically acceptable salt or solvate thereof, is administered in a single or plurality of doses once per: week, two weeks, three weeks, four weeks, two months, three months, four months, five months, six months, seven months, eight months, nine months, ten months, eleven months, or twelve months. In some cases, the composition is administered once per three months. In some cases, the composition is administered once per four months. In some cases, the composition is administered once per five months. In some cases, the composition is administered once per six months. In some cases, the composition is administered once per seven months. In some cases, the composition is administered once per eight months. In some cases, the composition is administered once per nine months. In some cases, the composition is administered once per ten months. In some cases, the composition is administered eleven per three months. In some cases, the composition is administered once per twelve months. In some cases, the composition is administered weekly for no more than five weeks.

In some embodiments, a composition comprising compound A, or a pharmaceutically acceptable salt or solvate thereof, is administered in a total volume from about 0.5 mL to about 10 mL in a single or a plurality of doses. In some cases, the composition is administered in a total volume from about 0.5 mL to about 10 mL, from about 0.5 mL to about 9 mL, from about 0.5 mL to about 8 mL, from about 0.5 mL to about 7 mL, from about 0.5 mL to about 6 mL, from about 0.5 mL to about 5 mL, from about 0.5 mL to about 4 mL, from about 0.5 mL to about 3 mL, from about 0.5 mL to about 2 mL, from about 0.5 mL to about 1 mL, from about 1 mL to about 9 mL, from about 1 mL to about 8 mL, from about 1 mL to about 7 mL, from about 1 mL to about 6 mL, from about 1 mL to about 5 mL, from about 1 mL to about 4 mL, from about 1 mL to about 3 mL, from about 1 mL to about 2 mL, from about 1.5 mL to about 5 mL, from about 1.5 mL to about 4 mL, from about 1.5 mL to about 3 mL, from about 1.5 mL to about 2 mL, from about 2 mL to about 5 mL, from about 2 mL to about 4 mL, or from about 2 mL to about 3 mL. In some cases, the total volume of composition does not exceed about 10 mL, does not exceed about 9 mL, does not exceed about 8 mL, does not exceed about 7 mL, does not exceed about 6 mL, does not exceed about 5 mL, does not exceed about 4 mL, does not exceed about 3 mL, does not exceed about 2 mL, or does not exceed about 1 mL. In some cases, the total volume of composition is about 10 mL, is about 9 mL, is about 8 mL, is about 7 mL, is about 6 mL, is about 5 mL, is about 4 mL, is about 3 mL, is about 2 mL, or is about 1 mL.

In some embodiments, a composition comprising compound A, or a pharmaceutically acceptable salt or solvate thereof, is administered at a concentration of compound from about 50 μg/mL to about 1000 μg/mL, from about 50 μg/mL to about 900 μg/mL, from about 50 μg/mL to about 800 μg/mL, from about 50 μg/mL to about 700 μg/mL, from about 50 μg/mL to about 600 μg/mL, from about 50 μg/mL to about 500 μg/mL, from about 50 μg/mL to about 400 μg/mL, from about 50 μg/mL to about 300 μg/mL, from about 50 μg/mL to about 200 μg/mL, from about 50 μg/mL to about 100 μg/mL, about 100 μg/mL to about 1000 μg/mL, from about 100 μg/mL to about 900 μg/mL, from about 100 μg/mL to about 800 μg/mL, from about 100 μg/mL to about 700 μg/mL, from about 100 μg/mL to about 600 μg/mL, from about 100 μg/mL to about 500 μg/mL, from about 100 μg/mL to about 400 μg/mL, from about 100 μg/mL to about 300 μg/mL, or from about 100 μg/mL to about 200 μg/mL of the compound. In some cases, the composition is administered at a composition of compound of at least about 100 μg/mL, about 150 μg/mL, about 200 μg/mL, about 250 μg/mL, about 300 μg/mL, about 350 μg/mL, about 400 μg/mL, about 450 μg/mL, or about 500 μg/mL of the compound.

### Combination Treatment

The compounds and compositions of the present invention can be used in combination with other components suitable for ameliorating arthritis or joint injury. In some embodiments, the composition can further comprise an additional compound which is therapeutically effective for the treatment of arthritis or joint injury and/or the symptoms associated with arthritis or joint injury in a mammal. In some embodiments, the composition can also include a non-steroidal anti-inflammatory drug (NSAID), an analgesic, a glucocorticoid, an angiopoietin-like 3 protein (ANGPTL3) or chondrogenic variant thereof, oral salmon calcitonin, SD-6010 (iNOS inhibitor), vitamin D3 (choliecalciferol), collagen hydrolyzate, FGF18, BMP7, avocado soy unsaponifiables (ASU) or hyaluronic acid. ANGPTL3 is described in more detail in WO201 1/008773 (incorporated herein in its entirety). In some embodiments, the composition includes an agent with anti-inflammatory activity. In some embodiments, the composition includes an apoptosis modulator. In certain embodiments, the apoptosis modulator is a caspase inhibitor. One non-limiting example of an apoptosis/caspase inhibitor is emricasan. In some embodiments, the composition includesan iNOS inhibitor. One non-limiting example of an iNOS inhibitor is SD-6010.

NSAIDS include, but are not limited to, aspirin, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, nabumetone, diclofenac, piroxicam, meloxicam, tenoxicam, droxicam, lornoxicam, isoxicam, mefenamic acid, meclofenamic acid, flufenamic acid, tolfenamic acid, celecoxib, parecoxib, etoricoxib, lumiracoxib, and firocoxib.

Analgesics include, but are not limited to, acetaminophen and opioids (narcotics). Opioids include, but are not limited to, dextropropoxyphene, codeine, tramadol, tapentadol, anileridine, alphaprodine, pethidine, hydocodone, morphine, oxycodone, methadone, diamorphine, hydromorphone, oxymorphone, levorphanol, 7-hydroxymitragynine, buprenorphine, fentanyl, sufentanil, bromadol, etorphine, dihydroetorphine, and carfentanil.

Glucocorticoids include, but are not limited to, hydrocortisone, cortisone, prednisone, prednisolone, methylprednisolone, dexamethasone, betamethasone, triamcinolone, beclometasone, or fludrocortisones.

Compound A, or a pharmaceutically acceptable salt or solvate thereof, may be used in combination with one or more compounds which are therapeutically effective for the treatment of arthritis or joint injury and/or the symptoms associated with arthritis or joint injury. Such additional compounds may be administered, by a route and in an amount commonly used therefore, contemporaneously or sequentially with a compound disclosed herein. When a compound disclosed herein is used contemporaneously with one or more such additional compounds, a pharmaceutical composition in unit dosage form containing such other drugs and the compound of the present invention is preferred. However, the combination therapy may also include therapies in which the compound disclosed herein and one or more additional compounds are administered on different overlapping schedules. It is also contemplated that when used in combination with one or more additional compounds, the compounds may be used in lower doses than when each is used singly.

The above combinations include combinations of compound A, or a pharmaceutically acceptable salt or solvate thereof not only with one compound which is therapeutically effective for the treatment of arthritis or joint injury and/or the symptoms associated with arthritis or joint injury, but also with two or more such compounds. Likewise, compounds disclosed herein, either in combination with a compound which is therapeutically effective for the treatment of arthritis or joint injury and/or the symptoms associated with arthritis or joint injury or by themselves, may be used in combination with other drugs that are used in the prevention, treatment, control, or amelioration of osteoarthritis or joint injury or conditions associated with osteoarthritis or joint injury. Such other drugs may be administered, by a route and in an amount commonly used therefore, contemporaneously or sequentially with a compound disclosed herein. When compound A disclosed herein is used contemporaneously with one or more other drugs, a pharmaceutical composition containing such other drugs in addition to the compound of the present invention is preferred. Accordingly, the pharmaceutical compositions of the present invention also include those that also contain one or more other active ingredients, in addition to a compound disclosed herein. The weight ratio of the compound disclosed herein to the second active ingredient may be varied and will depend upon the effective dose of each ingredient. Generally, an effective dose of each will be used.

### Administration of Pharmaceutical Composition

In one aspect, a composition comprising compound A, or a pharmaceutically acceptable salt or solvate thereof is administered by intra-articular injection. In some cases, the composition is administered to the knee. In some embodiments, excess fluid is aspirated from the knee prior to injection of the composition. Ultrasound may be used to guide the procedure as necessary. The route of IA injection may differ per joint, i.e. in the case of IA injection into the knee the synovial volume and mobility of the knee joint will differ from the hip or spinal joints.

### EXAMPLES

### Example 1: Human Chondrocyte Differentiation Assay

Human MSCs (50,000) were plated into each well of a 96-well plate and cultured overnight. Compound A (in DMSO solution) was added to the cells at a final concentration of 104, and the cells were cultured for 7 days at 5% CO₂, 37° C. The cells were fixed with 10% formalin solution at room temperature for 10 min, and immunostained using antibodies specific for type II collagen (Abcam), Sox9 (Santa Cruz) and cartilage oligomeric matrix protein (COMP, Santa Cruz), and fluorescently labeled secondary antibodies (Li-Cor). The total intensity of the staining was measured using Oddyssey CLx imaging system (Li-Cor). Vehicle (DMSO) was used as control to determine the basal level of chondrocyte differentiation. The result is shown in Table 1 [A: >50% increase in staining intensity compared to vehicle control; B: 30-50% increase in staining intensity compared to vehicle control].

TABLE 1

| | Biological |
|---|---|
| Structure | Activity |
| 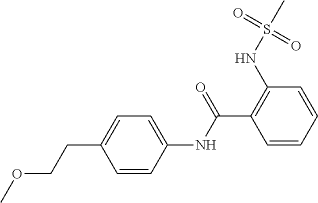 [SMILES: COCCc1ccc(NC(=O)c2ccccc2NS(C)(=O)=O)cc1] | A |
| Compound A | |

### Example 2: Compound A Cell Viability Assay

Human MSCs, chondrocytes, osteoblasts and synoviocytes are plated into 384-well plates at 10,000 cells per well. Compound A is added at a final concentration of 10004. The cells are cultured for 48 h. Cell viability is analyzed by Cell Titer-Glo (Promega) assay using EnVision plate reader (PerkinElmer). Apoptosis activity is analyzed by Caspase 3/7-Glo (Promega) assay using EnVision plate reader (PerkinElmer).

### Example 3: Compound A PK Study Via Intra-Articular Injection in Rats

A 30 μl-compound A solution (100 μM in PBS containing 0.1% DMSO) is injected into the articular space of the right knee of each rat. The animals are bled at 1, 3, 4, 6, 7, 8, 9, and 10 hours post-injection. The animals are terminated at 2 or 12 hours post-dose. Plasma and joint lavage of the injected knees are collected. The quantities of the injected compounds are analyzed using LCMS.

### Example 4: Rat Medial Meniscal Tear (MMT) Osteoarthritis (OA) Models

The medial meniscus of the right knee of each animal is surgically torn to induce OA. Dosing of the compound A solution (30 μl of 100 μM in PBS containing 0.1% DMSO) is begun 7 days post-surgery at one dose per week for three weeks. Body weights and gait deficits are monitored weekly right before dosing. Animals are terminated at day 28 post-surgery. The joints of the operated knees are processed and histochemically stained for cartilage, and the cartilage is evaluated.

Following 4-6 days in 5% formic acid decalcifier, the operated joints are cut into two approximately equal halves in the frontal plane and embedded in paraffin. Three sections are cut from each operated right knee (g1-8) at approximately 200 μm steps and stained with toluidine blue. Left knees of group 1 and right knees from group 9 have a single section prepared and stained with toluidine blue.

All three sections of each operated knee are analyzed microscopically. The worst-case scenario for the two halves on each slide is determined for general cartilage degeneration, proteoglycan loss, collagen damage, and osteophyte formation. The values for each parameter are then averaged across the three sections to determine overall subjective scores.

In addition, for some parameters (noted below), regional differences across the tibial plateau are taken into consideration by dividing each section into three zones (1-outside, 2-middle, 3-inside). In the surgical OA model, the outside (z1) and middle (z2) thirds are most severely affected, and milder changes are present on the inside third (z3). When zones are scored individually, scores are assigned based on percent area of the zone affected. Zone areas are delineated using an ocular micrometer.

The following parameters are measured and/or scored:

General cartilage degeneration includes the important parameters of chondrocyte death/loss, proteoglycan loss, and collagen loss or fibrillation. Cartilage degeneration in the tibia is scored none to severe (numerical values 0-5) for each zone using the following criteria:
0=no degeneration
1=minimal degeneration, within the zone 5-10% of the matrix appears non viable as a result of significant chondrocyte loss (greater than 50% of normal cell density). PG loss is usually present in these areas of cell loss and collagen matrix loss may be present.
2=mild degeneration, within the zone 11-25% of the matrix appears non viable as a result of significant chondrocyte loss (greater than 50% of normal cell density). PG loss is usually present in these areas of cell loss and collagen matrix loss may be present.
3=moderate degeneration, within the zone 26-50% of the matrix appears non viable as a result of significant chondrocyte loss (greater than 50% of normal cell density). PG loss is usually present in these areas of cell loss and collagen matrix loss may be present.
4=marked degeneration, within the zone 51-75% of the matrix appears non viable as a result of significant chondrocyte loss (greater than 50% of normal cell density). PG loss is usually present in these areas of cell loss and collagen matrix loss may be present.
5=severe degeneration, within the zone 76-100% of the matrix appears non viable as a result of significant chondrocyte loss (greater than 50% of normal cell density). PG loss is usually present in these areas of cell loss and collagen matrix loss may be present.
   In some cases, image analysis may be used to determine the exact % of matrix viability and/or loss in each zone or in selected zones so that absolute % rather than scores (0-5) can be compared. A 3-zone sum for cartilage degeneration is calculated in addition to expressing the data for each zone.

The same process is applied to evaluation of the femoral cartilage with the exception that lesions are not analyzed based on zones since the lesions are not generally distributed over the surface in a zonal pattern. The total width of the load-bearing surface (approximately 2000 μm for the femur) is determined and the above criteria is applied to the most severely affected ⅓, ⅔ or 3/3. For example, if ⅓ of the total area (lesion may be in the center of the plateau covering about 667 μm) has minimal degeneration (5-10% of total area has loss of chondrocytes and/or matrix), a score of 1 is assigned. If that minimal degeneration extends over the entire surface (3/3) then the score is 3. If the entire femoral cartilage is absent as a result of severe diffuse degeneration, then the score is 15.

In addition to this overall cartilage degeneration score, collagen matrix damage is scored separately in order to identify more specific effects of agents. Collagen damage across the medial tibial plateau (most severely affected section of the two halves) is quantified by measuring the total width of the following:
Any damage (fibrillation ranging from superficial to full thickness loss).
Severe damage (total or near total loss of collagen to tidemark, >90% thickness)
Marked damage (extends through 61-90% of the cartilage thickness)
Moderate damage (extends thru 31-60% of the cartilage thickness)
Mild damage (extends through 11-30% of the cartilage thickness)
Minimal damage (very superficial, affecting upper 10% only)

In addition to the above subjective general cartilage scoring, two cartilage degeneration width measurements are taken:
Total Tibial Cartilage Degeneration Width (μm) is a micrometer measurement of total extent of tibial plateau affected by any type of degeneration (cell loss, proteoglycan loss or collagen damage). This measurement extends from the origination of the osteophyte with adjacent cartilage degeneration (outside ⅓) across the surface to the point where tangential layer and underlying cartilage appear histologically normal.
Substantial Cartilage Degeneration Width (μm) reflects areas of tibial cartilage degeneration in which both chondrocyte and proteoglycan loss extend through greater than 50% of the cartilage thickness. In general, the collagen damage is mild (25% depth) or greater for this parameter but chondrocyte and proteoglycan loss extend to at least 50% or greater of the cartilage depth.

A micrometer depth of any type of lesion (both chondrocyte and proteoglycan loss, but may have good retention of collagenous matrix and no fibrillation), expressed as a ratio of depth of changed area vs. depth to tidemark, is taken in the area of greatest lesion severity in each of the three zones across the tibial surface at the midpoint of the zone. This measurement is the most critical analysis of any type of microscopic change present. The denominator can serve as an average measure of cartilage thickness in each of the three zones for comparison of anabolics when measures are taken at the midpoint of the zone.

Scoring of the osteophytes and categorization into small, medium and large is done with an ocular micrometer. Marginal zone proliferative changes have to be >200 μm in order to be measured and designated as osteophytes. Scores are assigned to the largest osteophyte in each section (typically found in the tibia) according to the following criteria:
1=small up to 299 μm
2=moderate 300-399 μm
3=large 400-499 μm
4=very large 500-599
5=very large ≥600

The actual osteophyte measurement (tidemark to furthest distance point extending toward synovium) is also recorded.

The femoral cartilage degeneration score and the three-zone sum of the tibial cartilage degeneration scores (mean of three levels) are summed to create a total cartilage degeneration score. The mean osteophyte score for each joint is added to this value to produce a total joint score.

### Image Analysis

In order to quantify and compare the cartilage matrix preservation, cartilage area measurements are taken from the most severely affected section of each animal. Photomicrographs are taken with a CoolSNAP-Pro microscope camera and loaded into ImagePro Plus software. The following measurements are taken from tracings of these photomicrographs, four per page, which are included in the report:
Total area from the tidemark to the surface (or projected surface in degenerated areas) over 9 cm (photomicrograph) of the tibial plateau, measured from the inner edge of the osteophyte
Area of non-viable matrix (cartilage with less than 50% chondrocytes, proteoglycan, and intact collagen) and no matrix within the total area
Area of no matrix within the total area

The area of non-viable matrix is subtracted from the total area to get the area of viable matrix, and the area of no matrix is subtracted from the total area to get the area of any matrix (collagen matrix with or without chondrocytes and proteoglycan). These two values are then compared back to the total area to derive the percent viable matrix area and the percent any matrix area, which are compared between groups. Five left knees from the vehicle group are included in this process as normal controls. This process may be used to analyze the entire surface or selected zones depending on lesion severity and apparent treatment effects.

Synovial reaction, if abnormal, is described (should be mainly fibrosis) and characterized with respect to inflammation type and degree but is not included in the OA score.

Damage to the calcified cartilage layer and subchondral bone (worst case scenario for all sections) is scored using the following criteria:
0=No changes
1=Increased basophilia at tidemark, no fragmentation of tidemark, no marrow changes or if present minimal and focal
2=Increased basophilia at tidemark, minimal to mild focal fragmentation of calcified cartilage of tidemark, mesenchymal change in marrow involves ¼ of total area but generally is restricted to subchondral region under lesion
3=Increased basophilia at tidemark, mild to marked focal or multifocal fragmentation of calcified cartilage (multifocal), mesenchymal change in marrow is up to ¾ of total area, areas of marrow chondrogenesis may be evident but no major collapse of articular cartilage into epiphyseal bone (definite depression in surface)
4=Increased basophilia at tidemark, marked to severe fragmentation of calcified cartilage, marrow mesenchymal change involves up to ¾ of area and articular cartilage has collapsed into the epiphysis to a depth of 250 μm or less from tidemark (see definite depression in surface cartilage)
5=Increased basophilia at tidemark, marked to severe fragmentation of calcified cartilage, marrow mesenchymal change involves up to ¾ of area and articular cartilage has collapsed into the epiphysis to a depth of greater than 250 μm from tidemark
   In addition, measurements are made of the thickness of the medial synovial/collateral ligament repair in a non-tangential area of the section.

Growth plate thickness is measured in all knees on medial and lateral sides (2 measures/joint) at the approximate midpoint of the medial and lateral physis (assuming a non tangential area of the section).

### Example 5: Extraction and Quantitation of Compound A in Joint and Plasma Rat Samples

LC-MS/MS analysis for compound A, or a pharmaceutically acceptable salts or solvates thereof, were performed using an API 3000 equipped with an Agilent 1100 HPLC and a Leap Technologies autosampler. A HPLC Phenomenex 5 micron, 100 A Luna C18 (2) analytical column with dimensions of 2.0×50 mm (Part No. 00B-4252-B0) at a temperature of 30 C, flow rate of 0.6 mL/min, injection volume of 10 uL, and a 6.0 min run time was used. Mobile phase A1 was 0.1% formic acid in water and Mobile phase B1 was 0.1% formic acid in acetonitrile. The gradient was 90% A1/10% B1 at time 0; 90% A1/10% B1 at time 1.0 min; 10% A1/90% B1 at time 2.0 min; 10% A1/90% B1 at time 4.0 min; 90% A1/10% B1 at time 4.10 min; 90% A1/10% B1 at time 6.0 min. Analytes and internal standard quantitation were performed using Multiple Reaction Monitoring (MRM) quantitation method. Listed below are specific methods used to dose and measure exposure in plasma and the observed concentration in joint extract.

Rat Plasma Samples: Calibration standard curve was prepared by serial dilution of a concentrated, spike solution of the compound in control rat plasma. Calibration standards and rat plasma samples were prepared via protein precipitation by adding aliquots of Acetonitrile and internal standard to each aliquot of standards and samples. Following vortex mixing and centrifugation, aliquots of the supernatants from each standards and samples were diluted with formic acid in water, mixed and injected. All plasma samples collected after IA dosing (starting at t=0, 0.5, 1, 2, 4, and 6 h) indicated no systemic exposure for any of the compounds listed in Table 2.

Rat Knee Joint Samples: Calibration standard curve was prepared by serial dilution of a concentrated, spike solution of the compound in internal standard diluents. Internal standard diluent was prepared by dissolving the internal standard compound at a certain concentration in acetonitrile. Rat knee joint samples for each time points were individually crushed and transferred into each centrifuge tube and added 1.0-mL of internal standard diluent. Each centrifuge tube was vortexed and centrifuged for 30 minutes. From each tube, supernatant was removed and injected onto the column for analysis. In addition, plasma samples were obtained by retro-orbital bleeds into heparin coated tubes and stored at −80 C and later processed by analogy to the protocol described above for rat plasma samples.

Compound administration and tissue processing: 30 uL of 100 μM compound A solution (PBS with 0.1% DMSO) was injected into the intra-articular space of the right hinder knee of each animal. The animals were euthanized at indicated time points (0 hr, 0.5 hr, 1 hr, 2 hr, 4 hr and 6 hr). Four animals were used for each timepoint. The injected knee joints were harvested, flash freeze in liquid nitrogen. The whole joints were grounded into powder while frozen, mixed with 1 mL internal standard-containing acetonitrile, incubated at 4° C. overnight, vortexed and centrifuged for 30 min. The supernatant from each sample was analyzed using LC-MS/MS. Data shown in Table 2 indicates the observed concentration in knee extract. ND=Not determined.

TABLE 2

| | Concentration observed in extract (ng/mL) | | | | | |
|---|---|---|---|---|---|---|
| Compound | T = 0 h | T = 0.5 h | T = 1 h | T = 2 h | T = 4 h | T = 6 h |
| A | 925.5 | 50.6 | 4.4 | 0 | ND | ND |

### Example 6A: Composition of Compound A

To prepare a parenteral pharmaceutical composition suitable for administration by injection, 100 mg of compound A, or a pharmaceutically acceptable salt or solvate thereof, is dissolved in DMSO and then mixed with 10 ml of 0.9% sterile saline solution. The mixture is incorporated into a dosage unit suitable for administration by injection.

### Example 6B: Composition of Compound A

The batch formula to manufacture 1.0 L of compound A injection, 200 μg/mL is provided in Table 3.

TABLE 3
Batch formula

| | Quantity | Quantity |
|---|---|---|
| Ingredient | (mg/5 mL vial) | (g)/1.0 L |
| Compound A | 1.0 | 0.20 |
| Disodium phosphate | 53.5 | 10.7 |
| dodecahydrate² | | |
| Sodium Chloride | 15.0 | 3.0 |
| 2M, Hydrochloric Acid Solution | Adjust to pH 7.9 | Adjust to pH 7.9 |
| PEG 3350 | 150 | 30.0 |
| Polysorbate 80 | 25.0 | 5.0 |
| Benzyl Alcohol | 47.0 | 9.4 |
| Water for Injection | QS to 5.0 mL¹ | QS to 1000 mL |

¹Vials were filled with 10% overage to allow for complete withdrawal of the labeled volume.
²The quantity is based on the amount used to prepare the phosphate buffer. The final quantity may be less based on the amount of phosphate buffer solution used to QS to final volume.

### Preparation of Phosphate Buffer Solution

To make buffer solution, disodium phosphate dodecahydrate was weighted and added to a beaker containing Water for Injection (WFI). The solution was stirred until completely dissolved. Sodium chloride was weighted and added to the buffer solution. The solution was stirred until completely dissolved. The pH of the solution was measured and sufficient HCl solution was added to achieve pH 7.9. QS with WFI. The pH was measured and adjusted if required to achieve pH 7.9.

### Preparation of Final Bulk Drug Solution

PEG-3350 was weighted into a tared jacketed beaker. The balance was tared and polysorbate 80 was weighted into the jacketed beaker. The jacketed beaker was attached to a water bath and set to approximately 70° C. to melt the PEG-3350. Compound A was weighted directly into a vial. Benzyl alcohol was added. A magnetic stirrer was added in the vial and the solution was stirred until compound A completely dissolved. The buffer solution was weighted to use as a rinse. The compound A/benzyl alcohol solution was added to the PEG-3350/polysorbate 80 solution, rinsing the drug vial with the reserved buffer solution and added to the drug solution. The resulting solution was stirred for approximately 10 minutes. The heat was turned heat off and the stirring was continued for approximately 10 minutes. The amount of buffer left to add was calculated and weighted into a beaker and added to the final bulk. Cooled to room temperature.

### Fill/Finish Vials

The solution was filtered through one 0.45 μm filter and two 0.22 μm, PVDF filters in series into a Schott bottle inside the laminar flow cabinet. The sterilized vials were filled. Stopper and crimp each vial in the laminar flow cabinet.

### Example 7: Stability Data

Compound A injection 200 μg/mL (from example 6B) has been placed on stability. Vials containing 5 mL of compound A injection were placed on storage at −20° C., 5° C., 25° C. and 40° C.

### Analytical Procedures

Appearance: Compound A Injection was examined under ambient light for its clarity, color and absence of foreign matter.

### Identification by HPLC: Purity/Related Substances by HPLC

### Column: Halo C18, 150×4.6 mm, 2.7 μm

Column temperature: 30° C.
Detection wavelength: UV@210 nm
Autosampler temperature: 25° C. (controlled ambient)
Flow rate: 1 mL/min
Injection volume: 5 μl
Mobile phase A: water:acetonitrile: Heptaflourobutyric acid (HFBA), 95:5:0.005, v/v/v
Mobile phase B: water:acetonitrile:HFBA, 5:95:0.0075, v/v/v
Diluent: water:acetonitrile, 50:50, v/v

### Gradient

| Time (min) | % A | % B |
|---|---|---|
| 0 | 100 | 0 |
| 3.5 | 100 | 0 |
| 13.5 | 50 | 50 |
| 21.0 | 35 | 65 |
| 23.5 | 0 | 100 |
| 24.0 | 0 | 100 |
| 24.1 | 100 | 0 |
| 30.0 | 100 | 0 |

Run time: 30 minutes
Integration time: 25 minutes

Sample preparation: Transfer a portion of drug product to an HPLC vial and inject neat.

Quantitation: The level of quantitation is >0.05%

pH: USP <791> method was followed.

Osmolality: USP <785> methodology was followed

Particulate Matter: USP <788> methodology was followed.

Sterility: USP <71> methodology was followed.

Bacterial Endotoxins: USP <85> methodology was followed.

Content Uniformity: USP <905> methodology was followed.

The stability data tables are provided in Table 4 through Table 10.

TABLE 4
Stability Data for Appearance, Osmolality and pH

| | | | Osmolality | |
|---|---|---|---|---|
| Timepoint/storage | | Appearance | mOsmkg⁻¹ | pH |
| Initial (T = 0) | | Clear, colorless solution, free of visible | 300 | 7.9 |
| | | particulates | | |
| −20° C. | T = 2 wk | Clear, colorless solution, free of visible | — | 7.9 |
| | | particulates | | |
| | T = 1 mo | Clear, colorless solution, free of visible | | 7.9 |
| | | particulates | | |
| | T = 2 mo | Clear, colorless solution, free of visible | | 7.8 |
| | | particulates | | |
| | T = 3 mo | Clear, colorless solution, free of visible | 300 | 7.9 |
| | | particulates | | |
| 5° C. | T = 2 wk | Clear, colorless solution, free of visible | — | 7.9 |
| | | particulates | | |
| | T = 1 mo | Clear, colorless solution, free of visible | | 7.9 |
| | | particulates | | |
| | T = 2 mo | Clear, colorless solution, free of visible | | 7.9 |
| | | particulates | | |
| | T = 3 mo | Clear, colorless solution, free of visible | 303 | 8.0 |
| | | particulates | | |
| 25° C./60% RH | T = 2 wk | Clear, colorless solution, free of visible | — | 7.9 |
| | | particulates | | |
| | T = 1 mo | Clear, colorless solution, free of visible | | 7.9 |
| | | particulates | | |
| | T = 2 mo | Clear, colorless solution, free of visible | | 7.9 |
| | | particulates | | |
| | T = 3 mo | Clear, colorless solution, free of visible | 303 | 8.0 |
| | | particulates | | |
| 40° C./75% RH | T = 2 wk | Clear, colorless solution, free of visible | | 7.9 |
| | | particulates | | |
| | T = 1 mo | Clear, colorless solution, free of visible | | 7.8 |
| | | particulates | | |
| | T = 2 mo | Clear, colorless solution, free of visible | | 7.9 |
| | | particulates | | |
| | T = 3 mo | Clear, colorless solution, free of visible | 308 | 7.9 |
| | | particulates | | |

TABLE 5
Stability data for Sub-visible Particulates

| | | Count/vial¹ | | |
|---|---|---|---|---|
| Timepoint/Storage | | ≥10 μm | ≥25 μm | Pass/Fail² |
| Initial (T = 0) | | 650 | 450 | PASS |
| −20° C. | T = 2 wk | 493 | 310 | PASS |
| | T = 1 mo | 30 | 0 | PASS |
| | T = 2 mo | 90 | 0 | PASS |
| | T = 3 mo | 97 | 0 | PASS |
| 5° C. | T = 2 wk | 107 | 33 | PASS |
| | T = 1 mo | 50 | 0 | PASS |
| | T = 2 mo | 77 | 0 | PASS |
| | T = 3 mo | 7 | 3 | PASS |
| 25° C./60% RH | T = 2 wk | 87 | 3 | PASS |
| | T = 1 mo | 87 | 0 | PASS |
| | T = 2 mo | 100 | 13 | PASS |
| | T = 3 mo | 23 | 3 | PASS |
| 40° C./75% RH | T = 2 wk | 123 | 13 | PASS |
| | T = 1 mo | 57 | 0 | PASS |
| | T = 2 mo | 150 | 0 | PASS |
| | T = 3 mo | 57 | 7 | PASS |

¹Where fill volume is 5 mL
²Number particles with diameter ≥10 μm per vial <6000 = PASS and number particles with diameter ≥25 μm per vial <600 = PASS

TABLE 6
Assay Stability Data

| | | Compound | | |
|---|---|---|---|---|
| | | A | Rec/theory¹ | Rec/T = 0² |
| Timepoint/Storage | | (μg/mL) | (%) | (%) |
| Initial (T = 0) | | 204.37 | 102.2 | — |
| −20° C. | T = 2 wk | 201.21 | 100.6 | 98.5 |
| | T = 1 mo | 202.21 | 101.1 | 98.9 |
| | T = 2 mo | 199.88 | 99.9 | 97.8 |
| | T = 3 mo | 202.94 | 101.5 | 99.3 |
| 5° C. | T = 2 wk | 201.20 | 100.6 | 98.4 |
| | T = 1 mo | 202.21 | 101.1 | 98.9 |
| | T = 2 mo | 200.06 | 100.0 | 97.9 |
| | T = 3 mo | 203.64 | 101.8 | 99.6 |
| 25° C./60% RH | T = 2 wk | 201.32 | 100.7 | 98.5 |
| | T = 1 mo | 202.18 | 101.1 | 98.9 |
| | T = 2 mo | 199.89 | 99.9 | 97.8 |
| | T = 3 mo | 202.62 | 101.3 | 99.1 |
| 40° C./75% RH | T = 2 wk | 201.21 | 100.6 | 98.5 |
| | T = 1 mo | 202.45 | 101.2 | 99.1 |
| | T = 2 mo | 200.14 | 100.1 | 97.9 |
| | T = 3 mo | 202.31 | 101.2 | 99.0 |

¹As percent of the theoretical concentration (200 ug/mL)
²As percent of T = 0 result

TABLE 7
Purity Stability Data

| Timepoint/Storage | | % Total Impurities¹ | RRT 0.78 |
|---|---|---|---|
| Initial (T = 0) | | 0.1 | 0.06 |
| −20° C. | T = 2 wk | 0.1 | 0.08 |
| | T = 1 mo | 0.1 | 0.13 |
| | T = 2 mo | 0.1 | 0.11 |
| | T = 3 mo | 0.2 | 0.15 |
| 5° C. | T = 2 wk | 0.2 | 0.19 |
| | T = 1 mo | 0.3 | 0.32 |
| | T = 2 mo | 0.4 | 0.36 |
| | T = 3 mo | 0.5 | 0.51 |
| 25° C./60% RH | T = 2 wk | 0.4 | 0.4 |
| | T = 1 mo | 0.6 | 0.59 |
| | T = 2 mo | 0.6 | 0.61 |
| | T = 3 mo | 1.0 | 0.92 |
| 40° C./75% RH | T = 2 wk | 0.6 | 0.58 |
| | T = 1 mo | 1.0 | 1.03 |
| | T = 2 mo | 1.1 | 0.99 |
| | T = 3 mo | 1.6 | 1.46 |

¹Sum of related substances ≥0.05%

TABLE 8
Stability at −20° C.

| Test | | | Stability Time Point (Months) | | | | | | | |
|---|---|---|---|---|---|---|---|---|---|---|
| (Specification) | Initial Release | | 1 | | 3 | | 6 | | 9 | |
| Appearance | Clear, | | Clear, | | Clear, | | Clear, | | Clear, | |
| (Clear, colourless | colorless | | colorless | | colorless | | colorless | | colorless | |
| solution) | solution | | solution | | solution | | solution | | solution | |
| pH | 7.9 | pH | 7.9 | pH | 7.9 | pH | 7.9 | pH | 7.9 | pH |
| (NLT 7.5 and NMT 8.2) | | | | | | | | | | |
| Osmolality | 298 | mOsm/kg | 297 | mOsm/kg | 297 | mOsm/kg | 299 | mOsm/kg | 298 | mOsm/kg |
| (250-350 mOsm/kg) | | | | | | | | | | |

| Volume in Container | 4.8 | mL | NR | NR | NR | NR |
|---|---|---|---|---|---|---|
| (NLT 4.5 mL) | | | | | | |
| Identity (HPLC) | Complies | | Complies | Complies | Complies | Complies |
| Assay (HPLC) | 99.9% of | | 101.3% of | 101.0% of | 99.6% of | 99.6% of |
| (NLT 0.18 and NMT | label claim | | label claim | label claim | label claim | label claim |
| 0.22 mg/mL) | (0.20 mg/mL) | | (0.20 mg/mL) | (0.20 mg/mL) | (0.20 mg/mL) | (0.20 mg/mL) |
| Purity (HPLC) | 99.9% | | 100.0% | 100.0% | 99.6% | 100.0% |
| (NLT 95.0% main peak) | | | (99.95%) | (99.95%) | | (99.95%) |
| Related Substances (HPLC) | RRT | Area % | Area % | Area % | Area % | Area % |
| (NMT 5.0% total NMT 1.0%, | 0.83-0.84 | 0.06 | 0.05 | 0.05 | 0.06 | 0.05 |
| each individual (Report all | 1.28-1.29 | <LOQ | 0.05 | <LOQ | <LOQ | <LOQ |
| impurities ≥0.05%) | Total | 0.06 | 0.05 | 0.05 | 0.06 | 0.05 |

NR = not required.

TABLE 9
Stability at 2-8° C.

| Test | | | Stability Time Point (Months) | | | | | | | |
|---|---|---|---|---|---|---|---|---|---|---|
| (Specification) | Initial Release | | 1 | | 3 | | 6 | | 9 | |
| Appearance (Clear, | Clear, | | Clear, | | Clear, | | Clear, | | Clear, | |
| colourless solution) | colourless | | colourless | | colourless | | colourless | | colourless | |
| | solution | | solution | | solution | | solution | | solution | |
| pH (NLT 7.5 and NMT 8.2) | 7.9 | pH | 7.9 | pH | 7.9 | pH | 7.9 | pH | 7.9 | pH |
| Osmolality (250-350 mOsm/kg) | 298 | mOsm/kg | 297 | mOsm/kg | 297 | mOsm/kg | 297 | mOsm/kg | 299 | mOsm/kg |

| Volume in Container (NLT 4.5 mL) | 4.8 | mL | NR | NR | NR | NR |
|---|---|---|---|---|---|---|
| Identity (HPLC) | Complies | | Complies | Complies | Complies | Complies |
| Assay (HPLC) (NLT 0.18 and NMT | 99.9% of | | 101.7% of | 101.1% of | 99.6% of | 99.5% of |
| 0.22 mg/mL) | label claim | | label claim | label claim | label claim | label claim |
| | (0.20 mg/mL) | | (0.20 mg/mL) | (0.20 mg/mL) | (0.20 mg/mL) | (0.20 mg/mL) |
| Purity (HPLC) | 99.9% | | 100.0% | 100.0% | 99.9% | 100.0% |
| (NLT 95.0% main peak) | | | (99.95%) | (99.95%) | | (99.95%) |
| Related Substances (HPLC) | RRT | Area % | Area % | Area % | Area % | Area % |
| (NMT 5.0% total | 0.83-0.84 | 0.06 | 0.05 | 0.05 | 0.06 | 0.05 |
| NMT 1.0%, each | 1.28-1.29 | <LOQ | 0.05 | <LOQ | <LOQ | <LOQ |
| individual | Total | 0.06 | 0.05 | 0.05 | 0.06 | 0.05 |

NR: Not Required.

TABLE 10
Stability at 25° C./60% RH

| Test | | | Stability Time Point (Months) | | | | | | | |
|---|---|---|---|---|---|---|---|---|---|---|
| (Specification) | Initial Release | | 1 | | 3 | | 6 | | 9 | |
| Appearance (Clear, | Clear, | | Clear, | | Clear, | | Clear, | | Clear, | |
| colourless solution) | colourless | | colourless | | colourless | | colourless | | colourless | |
| | solution | | solution | | solution | | solution | | solution | |
| pH (NLT 7.5 and NMT 8.2) | 7.9 | pH | 7.9 | pH | 7.9 | pH | 7.9 | pH | 7.9 | pH |
| Osmolality (250-350 mOsm/kg) | 298 | mOsm/kg | 297 | mOsm/kg | 298 | mOsm/kg | 297 | mOsm/kg | 299 | mOsm/kg |

| Volume in Container (NLT 4.5 mL) | 4.8 | mL | NR | NR | NR | NR |
|---|---|---|---|---|---|---|
| Identity (HPLC) | Complies | | Complies | Complies | Complies | Complies |
| Assay (HPLC) (NLT 0.18 and NMT | 99.9% of | | 101.4% of | 101.2% of | 99.7% of | 99.8% of |
| 0.22 mg/mL) | label claim | | label claim | label claim | label claim | label claim |
| | (0.20 mg/mL) | | (0.20 mg/mL) | (0.20 mg/mL) | (0.20 mg/mL) | (0.20 mg/mL) |
| Purity (HPLC) | 99.9% | | 100.0% | 100.0% | 99.9% | 100.0% |
| (NLT 95.0% main peak) | | | (99.95%) | (99.95%) | | (99.95%) |
| Related Substances (HPLC) | RRT | Area % | Area % | Area % | Area % | Area % |
| (NMT 5.0% total | 0.83-0.84 | 0.06 | 0.05 | 0.05 | 0.06 | 0.05 |
| NMT 1.0%, each | 1.28-1.29 | <LOQ | <LOQ | <LOQ | <LOQ | <LOQ |
| individual | Total | 0.06 | 0.05 | 0.05 | 0.06 | 0.05 |

NR: Not Required.

### Example 8: Randomized, Double-Blind, Placebo-Controlled, Dose Escalation Study Evaluating the Safety, Tolerability, Pharmacokinetics, and Pharmacodynamics of Compound a Administered Via Intra-Articular Injection in Subjects with Osteoarthritis of the Knee

### Primary Objective

To assess the safety and tolerability of compound A when administered via intra-articular injection to the knee joint

### Primary Endpoint

Incidence, relatedness, severity, and duration of treatment emergent adverse events (TEAEs) Secondary Objectives

To identify any dose limiting toxicity and determine the maximum tolerated dose of compound A To determine pharmacokinetic properties of compound A in plasma

### Secondary Endpoints

Changes from baseline in clinical laboratory test results, vital signs, or electrocardiogram (ECG) results

Clinically significant findings on physical examinations

Pharmacokinetic parameters of compound A in plasma including: Maximum observed plasma concentration (Cmax), Dose-adjusted Cmax (Cmax/dose); Time to maximum observed plasma concentration (Tmax), Area under the plasma concentration vs. time curve from time zero to the last quantifiable concentration (AUC0-t), Dose-adjusted AUC0-t (AUC0-t/dose), AUC from time zero to infinity (AUC0-∞), Dose-adjusted AUC0-∞ (AUC0-∞/dose), Terminal elimination rate constant (a,z), Terminal half-life (t½), Apparent clearance (CL/F), and Volume of distribution (Vz/F)

### Exploratory Objectives

To assess changes in cartilage and osteoarthritis symptoms after administration of compound A

To collect and bank biospecimen samples for exploratory biomarker research

### Exploratory Endpoints

Serum levels of the N-propeptide of type IIA collagen (PIIANP) as a pharmacodynamics marker of collagen synthesis

Urinary excretion of C-terminal cross-linking telopeptide of type II collagen (CTX-II) as a pharmacodynamic marker of collagen degradation

Change from baseline in Whole-Organ Magnetic Resonance Imaging Score (WORMS) of the index knee

Change from baseline of the Western Ontario and McMaster Universities Osteoarthritis Index (WOMAC) Version 3.1 total score and the WOMAC pain and function subscale scores

Utilize banked biospecimen samples for exploratory research related to drug response in OA

### Study Design

This is a randomized, double-blind, placebo-controlled study to assess the safety, tolerability, pharmacokinetics, and pharmacodynamics of compound A when administered via intra-articular injection to subjects with osteoarthritis of the knee. All subjects will receive a 5 mL injection of compound A or placebo in the affected knee.

The study will consist of approximately 7 cohorts of subjects who will be randomized to receive compound A or placebo. Compound A will be provided as a solution with a concentration of 200 μg per mL. Compound A will be administered as a single dose to the following cohorts:

| | |
|---|---|
| DOSE | N PER COHORT |

| | | |
|---|---|---|
| 50 | μg per knee | 4 (3 - Active & 1 Placebo) |
| 100 | μg per knee | 4 (3 - Active & 1 Placebo) |
| 200 | μg per knee | 8 (6 - Active & 2 Placebo) |
| 400 | μg per knee | 8 (6 - Active & 2 Placebo) |

The decision to escalate to the next dose cohort will be made by the sponsor, based on the recommendation of the Data Safety Monitoring Board (DSMB), following a (blinded) review of all available safety information from Day 8 post-dose of the preceding dose cohort.

Once the safety and tolerability of single doses of compound A have been assessed, multiple-dose administration of compound A will be evaluated. The multiple-dose portion of the study will be initiated after a thorough review of the Day 29 safety data from the single-dose cohorts. During the multiple dose portion of the study, compound A will be administered as 4 weekly doses to the following cohorts:

| DOSE | N PER COHORT |
|---|---|
| 100 μg per knee per injection | 12 (9 - Active & 3 Placebo) |
| 200 μg per knee per injection | 12 (9 - Active & 3 Placebo) |
| 400 μg per knee per injection | 12 (9 - Active & 3 Placebo) |

The doses administered to subsequent cohorts in either the single or multiple dose portions of the study may be lowered if any safety or tolerability issues are identified which suggest that the planned doses may pose a risk to study participants. Additional dose groups may be added to the study depending on the observed safety and tolerability profile of compound A or if the pharmacokinetic data permit a higher than anticipated dose while remaining within a safe exposure limit based on the toxicokinetics and safety profile from the nonclinical toxicology studies. The study will be conducted at approximately 4 sites in the United States. Approximately 60 subjects will be randomized to participate in this trial.

### Statistical Methods

This is a Phase 1 study with a primary objective of assessing the safety and tolerability of compound A. The pharmacokinetics and pharmacodynamics of compound A will be evaluated as secondary and exploratory endpoints. The proposed size of each dose cohort was chosen to provide sufficient information to allow assessment of the safety and tolerability of compound A and identify any potential safety signals or dose limiting toxicity before proceeding with administration of higher doses. Safety and tolerability will be evaluated by summarizing treatment emergent adverse events (TEAEs), serious adverse events, clinical laboratory test results, vital sign measurements, and electrocardiogram (ECG) findings. No formal statistical tests will be conducted to assess the safety or tolerability of compound A.

All PK samples will be analyzed by LC-MS/MS using a validated, sensitive, specific method. Descriptive statistics for plasma concentrations by time point and by treatment group will include number of observations, arithmetic mean, standard deviation, arithmetic coefficient of variation (% CV), geometric mean, median, geometric % CV, minimum, and maximum. Using non-compartmental methods, the plasma concentration versus time data will be used to derive the following PK parameters: Cmax, Cmax/dose, Tmax, AUC0-t, AUC0-t/dose AUC0-∞, AUC0-∞/dose, λz, terminal t½, CL/F, and Vz/F. Descriptive statistics for PK parameters by treatment group will include number of observations, arithmetic mean, standard deviation, arithmetic coefficient of variation (% CV), geometric mean, median, geometric % CV, minimum, and maximum. Dose proportionality will be explored.

The pharmacodynamics (PD) of compound A will be evaluated by examining several exploratory endpoints, including PIIANP, CTX-II, pain and physical functioning as assessed by the WOMAC, and MR imaging data. The pre-treatment values for these endpoints will be compared to post-treatment measurements and both the absolute and percent change from baseline will be summarized. Descriptive statistics for PD endpoints by time point and by treatment group will include number of observations, arithmetic mean, standard deviation, arithmetic coefficient of variation (% CV), median, minimum, and maximum. An exploratory analysis of PK/PD endpoints may also be performed.

### Study Treatments

For this study, the investigational product is compound A or matching placebo. compound A or placebo will be administered at the following dose levels to the single dose cohorts:
50 μg per IA injection
100 μg per IA injection
200 μg per IA injection
400 μg per IA injection

Compound A or placebo will be administered once a week for 4 weeks at the following dose levels to the multiple dose cohorts:
100 μg per IA injection
200 μg per IA injection
400 μg per IA injection

Compound A will be supplied in amber glass vials as a sterile solution with a concentration of 200 μg per mL. Matching placebo will be provided in identical vials. Each vial will contain 5 mL of compound A or placebo. The vials will be packaged in boxes for shipment to investigative sites.

The investigational product (IP) labels will include the protocol number, the contents, the lot number, storage conditions, and an investigational use caution statement.

### Administration

Compound A or placebo will be administered via ultrasound-guided intra-articular injection in accordance with the standard of care procedures at the site. compound A or placebo should be administered by the Principal Investigator or another qualified physician trained in accepted techniques for delivering agents to the knee joint. Strict aseptic injection technique must be employed during administration of compound A or placebo. The physician should use his or her professional judgment to choose the best approach and injection site for the individual subject.

Excess fluid should be aspirated from the knee prior to injection of the investigational product. Ultrasound must be used to guide the procedure. The physician should save the ultrasound images documenting the needle placement until the close-out visit for this study. Each injection will consist of 5 mL of compound A or placebo. Subjects should be advised to avoid strenuous activity after receiving compound A or placebo and to manage pain in accordance with standard post-injection care instructions used at the site. Post injection flare, characterized by localized pain, may occur within several hours of an intra-articular knee joint injection. It usually resolves within 48 hours. Any instances of post injection flare should be reported as an adverse event.

### Example 9: Intra-Articular Injection of Compound A in Patients with Osteoarthritis of the Knee

Compound A, or a pharmaceutically acceptable salt or solvate thereof, is administered via intra-articular injection into the knees of patients diagnosed with osteoarthritis to promote cartilage repair. Patients receive one injection of compound A, or a pharmaceutically acceptable salt or solvate thereof, or multiple injections and may be treated one time or may receive treatment on a regular basis (once every three months, once every six months, once every nine months, or once a year). As another non-limiting example, dosing is weekly for no more than five weeks.

### Example 10: Effect of Dose Frequency of Compound A in a Rat Medial Meniscal Tear Model

### Reagents and Instruments

The vehicle used for compound A is 3% PEG3350, 0.5% Tween80, 30 mM Sodium Phosphate buffer at pH 7.8. The vehicle was prepared separately, then added to aliquots of compound A powder, and the mixture was then vortexed until it becomes a clear solution. Dosing solutions were prepared for doses of 3.5 and 0.35 μg/kg (100 μM and 10 μM respectively). Dosing volume was set to 30 μL/knee. Dosing solutions were prepared fresh weekly.

### Animal Model

Fourteen-week-old male Lewis rats (Charles River, Kingston, N.Y.) were in the weight range of 300-320 g at the start of the study

### Maintenance Conditions

Animals were housed in pairs in disposable microisolators (Innovive, Innocage IVC rat cages) with access to food (standard rodent chow, Picolab rodent, Newco) and water ad libitum. They were allowed to acclimate to the facility for one week prior to study enrolment in a housing room that was on a 12 h:12 h light cycle (6 am to 6 pm), with a temperature range of 70-72° F. and humidity range from 40% to 69%

### Surgery

Rats were sorted based on body weight into 8 study groups of 15 rats. They were surgerized and enrolled in a staggered fashion across one week (20 animals per day for 6 days). Briefly, animals were anesthetized with a ketamine/xylazine cocktail (50 mg/kg Ketaved—Henry Schein, and 5 mg/kg Xyalzine—AniSed respectively) by intraperitoneal injection, administered appropriate analgesics (Flunixin, 5 mg/kg subcutaneous injection), and the surgical site shaved and disinfected. A small incision was made on the medial right hind knee to expose the collateral ligament. The collateral ligament was transected and the joint capsule opened sufficiently to expose the medial meniscus. The meniscus was transected completely, then the skin incision was closed, and pressure applied to the wound to prevent hematoma development. Animals were allowed to recover on a heated blanket and were returned to their home cage. Sham procedures were carried out in a similar fashion, except the medial meniscus was not cut. All surgical procedures were performed aseptically and all procedures were approved by the institute IACUC. Animals were monitored daily for the following week for postoperative care prior to dosing. Any animals with atypical healing (bruising/hematoma) were removed from the study and replaced with spare animals.

Animals were enrolled into the following groups:

### Treatment Groups

A. Vehicle, dosed once weekly
B. compound A 3.5 μg/kg; 1.05 μg/knee (100 μM) dosed once weekly
C. compound A 3.5 μg/kg; 1.05 μg/knee (100 μM) dosed every other week
D. compound A 3.5 μg/kg; 1.05 μg/knee (100 μM) dosed once
E. compound A 0.35 μg/kg; 0.105 μg/knee (10 μM) dosed once weekly
F. compound A 0.35 μg/kg; 0.105 μg/knee (10 μM) dosed once every other week
G. compound A 0.35 μg/kg; 0.105 μg/knee (10 μM) dosed once

### H. Sham

| Group | Day 0 | Day 7 | Day 14 | Day 21 | Day 28 |
|---|---|---|---|---|---|
| A | Surgery | Dose | Dose | Dose | Takedown |
| B | Surgery | Dose | Dose | Dose | Takedown |
| C | Surgery | Dose | | Dose | Takedown |
| D | Surgery | Dose | | | Takedown |
| E | Surgery | Dose | Dose | Dose | Takedown |
| F | Surgery | Dose | | Dose | Takedown |
| G | Surgery | Dose | | | Takedown |
| H | Surgery | Dose | Dose | Dose | Takedown |

Animals were weighed weekly at the time of dosing (8-10 am), and a standardized dose of 30 μl of either vehicle or compound was administered via injection into the intra-articular space according to the regimens outlined above (groups A, B, E and H dosed once weekly, groups C and F dosed every other week, and groups D and G dosed once for the duration of the study). Animals were dosed using a Hamilton syringe with a 27 G needle attached. On study day 28, animals were euthanized, a terminal blood sample collected, and knees were harvested and placed in formalin jars for histology analysis and pathology scoring.

### Data Analysis and Statistical Methods

Analysis of the knee joint histology was performed by Bolder BioPATH. A variety of measurements to characterize the lesions were performed to score the joints based on lesion depth, severity, and overall size.

### Results

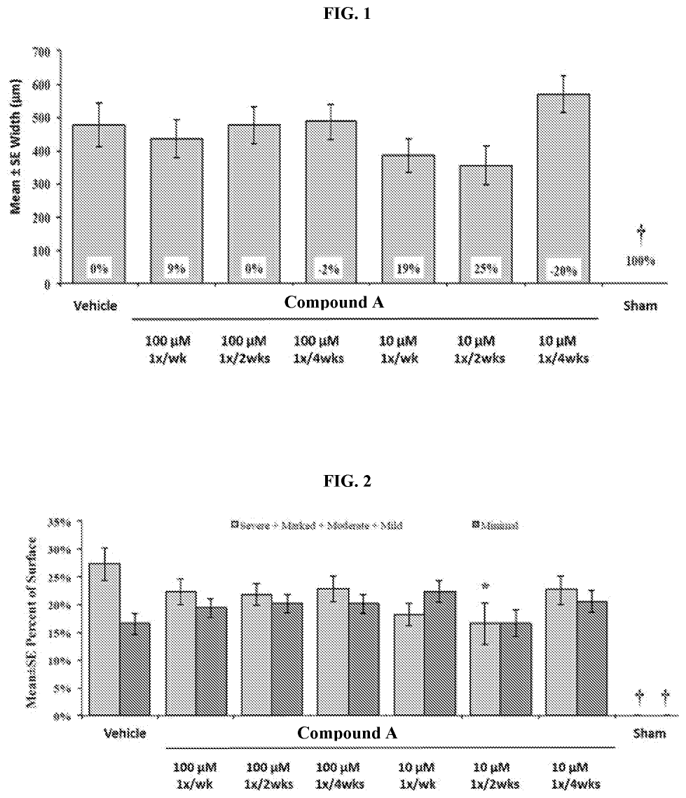

Throughout the study, animals showed no adverse effects with treatment of compound A at any dosing schedule (once weekly, once every other week, once per study) or any of the dosing concentrations tested (100 μM or 10 μM). Histological analysis revealed that treatment with 10 μM (0.35 μg/kg; 0.105 μg/knee) of compound A, when given once weekly or once every other week, was associated with improvement in substantial cartilage degeneration width (FIG. 1: Lewis rats were administered doses of compound A at 100 μM (3.5 μg/kg; 1.05 μg/knee) or 10 μM (0.35 μg/kg; 0.105 μg/knee) either once weekly, once every other week, or once throughout the duration of the study. At the end of the study, knees were harvested and fixed for sectioning. The width and depth of the cartilage lesions were measured and scored. The width of the lesion where greater than 50% of the cartilage is compromised or missing is plotted above. n=10-15 animals/group, † p<0.05 Student's t-test vs vehicle). Substantial cartilage degeneration is reflective of chondrocyte and proteoglycan loss greater than 50% of the cartilage depth. Though not statistically significant, treatment with compound A resulted in a trend towards an improvement in the most severe cartilage lesions where structural changes have occurred because of disease progression. Furthermore, when examining a range of severity outcomes in cartilage degeneration (ranging from minimal to severe cartilage damage), compound A dosed at 10 μM (0.105 μg/knee) once every other week showed a statistically significant improvement in combined cartilage degeneration widths with scores from mild to severe (FIG. 2: Lewis rats were administered doses of compound A at 100 μM (3.5 μg/kg; 1.05 μg/knee) or 10 μM (0.35 μg/kg; 0.105 μg/knee) either once weekly, once every other week, or once throughout the duration of the study. At the end of the study, animals were euthanized and knees harvested and fixed for histology. Lesions in the cartilage of the tibial plateau were measured using an ocular micrometer, and scored based on the severity of the lesions from minimal (<10% damaged), mild (11-25%), moderate (25-50%), marked (51-75%) and severe (76-100%). Once scored, the width of the tibial plateau is measured and the ratio of damaged cartilage to the total width of cartilage is calculated and presented. n=10-15 animals per group. * p<0.05 ANOVA (Dunn's post hoc test) vs. Vehicle. † p<0.05 Student's t-test vs. vehicle). Weekly doses of compound A at 10 μM (0.105 μg/knee) also demonstrated a trend towards a reduction in mild-to-severe degeneration widths.

The goal of this study was to explore various dosing frequencies for compound A within the context of a rat model of osteoarthritis. An intra-articular injection in a rodent knee can be challenging and invasive, given the size and volume restrictions in injecting into such a small joint. At some point, the injection process itself, when done too frequently, can lead to increased inflammation at the site of injection or potential iatrogenic damage from the syringe tip against the femoral/tibial bone surface. This could cause swelling of the joint and potentially result in a decrease in weight bearing and avoidance of using the knee entirely. This surgical model of osteoarthritis, unlike chemical methods of inducing joint disease such as the monoiodoacetate model, relies on the animal using the affected joint for cartilage damage to occur and degenerative lesions to form. Therefore, one must find a balance between delivering the optimal amount of compound while avoiding excessive manipulation of the joint itself which could confound interpretation of the results. In this study, animals were dosed once weekly, once every other week, and once for the duration of the 4-week study with two concentrations of compound A. The optimal efficacy was seen when compound A was dosed every other week at the lower of the two doses chosen. This suggests that dosing weekly may be too much manipulation of the joint in this model. In contrast, dosing once every four weeks did not demonstrate an effect on the progression of cartilage degeneration in this model. Dosing once every other week showed a significant reduction in the width of cartilage lesions where greater than 10% of the cartilage is damaged or lost. Additionally, the effect of compound A was mostly seen in an improvement in the most severe cartilage defects, where greater than 50% of the cartilage was lost. This is encouraging in that these regions of the lesion lack chondrocyte density (less than 50% normal cell density), where compound A may help to promote chondrocyte proliferation or prevent chondrocyte loss. In this rodent model of surgically induced osteoarthritis, compound A had the greatest efficacy when dosed intra-articularly once every two weeks.

### Example 11: Dose Range Study of Compound A in a Rat Medial Meniscal Tear Model

### Reagents and Instruments

The vehicle for compound A is 3% PEG3350, 0.5% Tween80, 30 mM Sodium Phosphate buffer at pH 7.8. The vehicle was prepared separately and then added to aliquots of compound A powder, after which the mixture was vortexed and becomes a clear solution. Dosing solutions were prepared for doses ranging from 30 μM to 0.3 μM. Dosing volume was set to 30 μL/knee. Dosing solutions were prepared fresh for each dose. Dosing solutions were verified at the end of the study. The positive control compound FGF-18 stock was reconstituted in 5 mM Tris, pH 8.0 as per manufacturer's instructions. The FGF-18 stock was further diluted in saline to a final concentration of 0.167 mg/mL. Fresh dilutions were prepared twice a week at the time of injection.

### Animal Model

Fourteen-week-old male Lewis rats (Charles River, Kingston, N.Y.) were in the weight range of 285-327 g.

### Maintenance Conditions

Animals were housed in pairs in disposable microisolators (Innovive, Innocage IVC rat cages) with access to food (standard rodent chow, Picolab rodent, Newco) and water ad libitum. They were allowed to acclimate to the facility for one week prior to study enrollment in a housing room that was on a 12 h:12 h light cycle (6 am to 6 pm) with a temperature range of 70-72° F., and humidity range from 40% to 69%.

### Surgery

Rats were sorted based on body weight into 8 study groups of 15 rats. They were surgerized and enrolled in a staggered fashion across one week (enroll 20 animals per day for 6 days). Animals were anesthetized with a ketamine/xylazine cocktail (50 mg/kg Ketaved—Henry Schein, and 5 mg/kg Xyalzine AniSed, respectively) by intraperitoneal injection, administered appropriate analgesics (Flunixin, 5 mg/kg subcutaneous injection), and the surgical site shaved and disinfected. A small incision was made on the medial right hind knee to expose the collateral ligament. The collateral ligament was transected and the joint capsule opened sufficiently to expose the medial meniscus. The meniscus was transected completely, then the skin incision was closed, and pressure applied to the wound to prevent hematoma development. Animals were allowed to recover on a heated blanket and were returned to their home cages. Sham procedures were carried out in a similar fashion except the medial meniscus was not cut. All surgical procedures were performed aseptically and all procedures were approved by the IACUC. Animals were monitored daily for the following week for postoperative care prior to dosing. Any animals with atypical healing (bruising/hematoma) were removed from the study and replaced with spare animals. Animals were enrolled into the following groups:

### Treatment Group

A. Vehicle, dosed once every other week
B. compound A 1.05 μg/kg; 0.315 μg/knee (30 μM) dosed once every other week
C. compound A 0.35 μg/kg; 0.105 μg/knee (10 μM) dosed once every other week
D. compound A 0.105 μg/kg; 0.0315 μg/knee (3 μM) dosed once every other week
E. compound A 0.35 μg/kg; 0.0105 μg/knee (1 μM) dosed once every other week
F. compound A 0.0105 μg/kg; 0.00315 μg/knee (0.3 μM) dosed once every other week
G. FGF-18 (5 μg/injection) dosed twice a week

### H. Sham

| Group | Day 0 | Day 7 | Day 14 | Day 21 | Day 28 |
|---|---|---|---|---|---|
| A | Surgery | Dose | | Dose | Takedown |
| B | Surgery | Dose | | Dose | Takedown |
| C | Surgery | Dose | | Dose | Takedown |
| D | Surgery | Dose | | Dose | Takedown |
| E | Surgery | Dose | | Dose | Takedown |
| F | Surgery | Dose | | Dose | Takedown |
| G | Surgery | Dose | Dose | Dose | Takedown |
| | | twice/week | twice/week | twice/week | |
| H | Surgery | Dose | | Dose | Takedown |

Animals were weighed weekly at the time of dosing (9 am-12 pm) and a standardized dose of 30 μl of vehicle or compound was administered via injection into the intra-articular space, according to the regimen outlined above. Groups A-F and H were dosed once every other week, and Group G received doses twice per week throughout the duration of the study. Animals were dosed using a Hamilton syringe with a 27 G needle attached. On study day 28, animals were euthanized, a terminal blood sample collected, and knees were harvested and placed in formalin jars for histology analysis and pathology scoring.

### Data Analysis and Statistical Methods

Analysis of the knee joint histology was performed by Bolder BioPATH. A variety of measurements were performed to score the joints based on lesion depth, severity, and overall size.

### Results

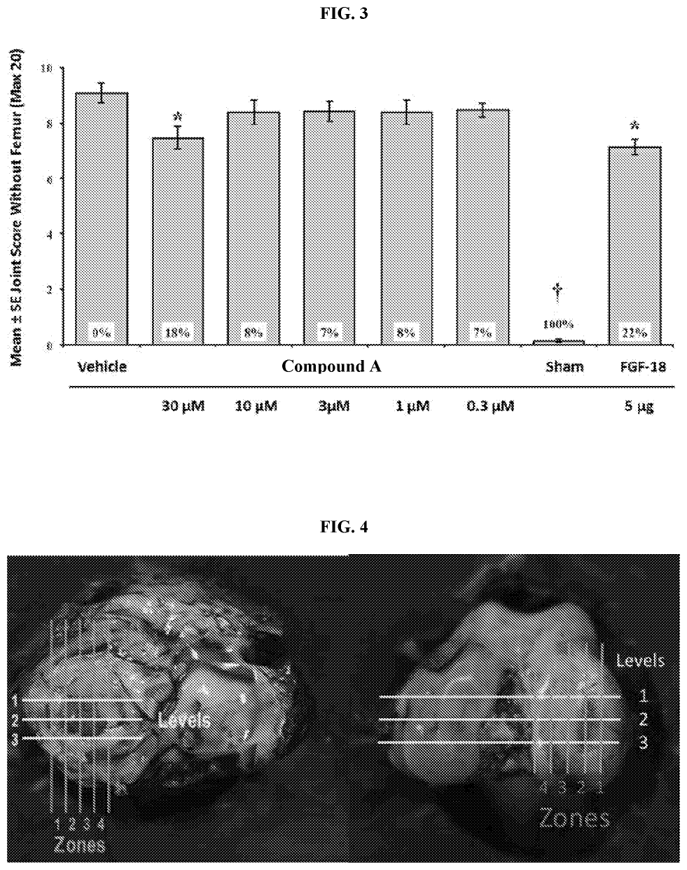

Throughout the study, animals showed no adverse effects with treatment of compound A at any of the doses tested (30, 10, 3, 1, 0.3 μM). Treatment with compound A at a dose of 30 μM (0.315 μg/knee) significantly improved total joint scores without the femur as compared to vehicle treated animals. (FIG. 3: Lewis rats were administered doses of compound A ranging from 30 μM (1.05 μg/kg; 0.315 μg/knee) to 0.3 μM (0.0105 μg/kg; 0.00315 μg/knee) once every other week throughout the duration of the study. At the end of the study, animals were euthanized and knees harvested and fixed for histology. The joint score is reflective of the sum of the tibial degeneration scores (based on measurements of the cartilage lesions) in combination with the osteophyte score. * p<0.05 Kruskal-Wallis test (Dunn's post hoc test) vs Vehicle. † p<0.05 Student's t-test vs. vehicle). Total joint scores without the femur include the cartilage degeneration and osteophyte scores across the affected tibia. The degree of improvement for the 30 μM dose of compound A was on par with FGF-18, the positive control reference compound.

The goal of this study was to determine the efficacious dose of compound A in a rat model of osteoarthritis. Animals were dosed via intra-articular injection once every other week with doses of compound A ranging from 30 μM (0.315 μg/knee) to 0.3 μM (0.00315 μg/knee). Efficacy was demonstrated at the 30 μM (0.315 μg/knee) dose, with a significant improvement in the total joint score excluding femur. The results of this study indicate that significant efficacy occurs with a dose of 30 μM (0.315 μg/knee) of compound A when administered once every other week in a rodent model of OA.

### Example 12: Efficacy of Compound A in a Canine Model of Osteoarthritis

Vials containing compound A powder were solubilized in 5% PEG300 and were vortexed to become clear solutions. To this solution, 95% saline was added to the final dosing concentration (0.0696 mg/mL 200 μM). The clear solution was then filtered in a 0.45 μm filter prior to intraarticular dose. Dosing solution was prepared fresh on the day of dosing.

### Bioanalytical Method

Acetonitrile, water (Optima, LC/MS Grade) and formic acid, 99+% (Optima, LC/MS Grade) were purchased from Fisher Scientific. Control Female Sprague Dawley rat plasma (Sodium Heparin, 0.2 u filtered) was purchased from Bioreclamation IVT. Chromatography HPLC column, Luna, 5 um C18 (2), 50×2.0 mm and its guard security cartridge and holder were purchased from Phenomenex. HPLC (1100 Series) was purchased from Agilent. The Mass Spectrometer system, API 3000 was purchased from SCIEX. Separation was performed on a reversed phase C18 column with a gradient elution using mobile phases of 0.1% formic acid in water (A) and 0.1% formic acid in acetonitrile (B) with flow rate at 0.6 mL/min. Ionization was achieved using electrospray in the negative mode (ESI−) for both compound A and internal standard Kartogenin. Multiple reaction monitoring (MRM) was used for drug quantification, Precursor to Product ion transition was 347.0>268.9 (m/z). MRM for internal standard Kartogenin was 316.0>272.0 (m/z).

Preparation of Standard Curve for compound A in Rat Plasma: standard curve for compound A was prepared in rat plasma by spiking compound A level in rat control plasma. Calibration Standard curves were generated in rat plasma ranging from 1.53 to 781.3 ng/mL. Standards and samples were both prepared by protein precipitation (using internal standard, Kartogenin or KGN at 250 ng/mL in cold acetonitrile) and centrifugation. Supernatant was then diluted with 0.1% formic acid in water solvent and injected onto LC-MS-MS system.

### Animal Model

Twelve to fifteen-month-old female naïve Beagle dogs were obtained from Marshall BioResouces (North Rose, N.Y.) with a weight range of 5.4 to 9.3 kilograms.

### Maintenance Conditions

All dogs were acclimated in the facility for 31 days prior to study start. Animals were prescreened prior to study enrollment for general health and body weight. All animals were healthy at the time of enrollment. Animals were housed in pens of 5 animals per pen, with 12 hour light/12 hour dark light cycle (6 am/6 pm). The rooms were ventilated with greater than 10 air changes per hour with at least 60% fresh air. Room temperatures were maintained as per SOPs dictated by ASI. Dogs were provided ad libitum access to food (ProLab: Animal Diet 5006) and water (chlorinated municipal tap water). The only exception to the feeding schedule was for periods of fasting prior to anesthesia.

### Surgery

Prior to surgical intervention, female Beagles were observed for several days to determine behavioral tendencies (high activity, lethargy, etc.). The goal was to sort and ensure that behavioral characteristics were shared among all groups, so that the activity level and use of the joints while not in regimented exercise would be uniform between groups. In addition to sorting by temperament, animals were also sorted by body weights, with equal distribution between study groups. Animals were acclimated to an exercise regimen of 1 hour of walking/running/playing prior to study start.

Animals were enrolled into the following 3 groups:

| Group | Number | Surgery | Treatment |
|---|---|---|---|
| 1 | 10 | Sham | Vehicle (5% PEG300/95% Saline) |
| 2 | 10 | Medial Meniscal | Vehicle (5% PEG300/95% Saline) |
| | | Tear | |
| 3 | 10 | Medial Meniscal | Compound A |
| | | Tear | (200 μM; 0.035 mg/knee) |

Animals were food deprived prior to surgery, anesthetized using propofol (6 mg/kg, IV), and maintained on isoflurane (3-4%) in oxygen (2 L/min) for the duration of the procedure (10-15 minutes). The right rear knee was shaved and aseptically prepared. A medial skin incision was made to expose the collateral ligament. A portion of the medial meniscus was cut (a wedge roughly half the width of the medial meniscus was removed). The joint capsule and subcutis were closed with sutures, and skin closed with wound clips. Sham animals were treated in the same manner except for the medial meniscus partial transection. All animals were allowed to recover for 10-11 days prior to dosing. Three days after surgery, all animals began an exercise regimen of one hour of continuous exercise (walking/running/playing), to ensure that the animals were using the surgerized leg and to allow for a more uniform development of lesions across a group of differently tempered dogs.

All animals were exercised 5 days a week for an hour for the duration of the study. At study day 10/11, animals began receiving weekly injections of either compound or vehicle into the surgerized joint. Blood samples were also taken pre-dose for all animals for compound determination. Animals received doses at day 10, 17, 24, 31, 38, and 45 post surgery. All intraarticular doses were standardized to 0.5 mL injections into the joint capsule via injection through the patellar ligament. In a subset of animals, a final dose PK was performed where animals were dosed and blood samples collected 0.5, 1, 2, 4, 8, 12 and 24 hours post dose. Summary data are presented in Table 11.

TABLE 11
Final dose PK of animals treated with an intra-articular
injection of 0.035 mg/knee of compound A

| Dose | | Cmax | Tmax | AUC 0-τ^{a} | Tₗₐₛₜ |
|---|---|---|---|---|---|
| (μg) | Animal | (ng/mL) | (h) | (h*ng/mL) | (h) |
| 35 | 1 | 2.26 | 0.5 | 3.58 | 4.0 |
| | 2 | 1.08 | 0.5 | 1.15 | 2.0 |
| | 3 | 1.54 | 0.5 | 1.87 | 2.0 |
| | 4 | 2.22 | 0.5 | 2.28 | 2.0 |
| | 5 | 2.64 | 0.5 | 3.14 | 2.0 |
| | 6 | 1.87 | 0.5 | 2.12 | 2.0 |
| | Mean | 1.94 | 0.5 | 2.36 | 2.3 |

Female Beagles underwent medial meniscal tear surgery to induce osteoarthritis. Animals were treated with either vehicle or compound A at a dose of 0.035 mg/knee (200 μM) once weekly via intra-articular injection. The study lasted for 6 weeks, and after the 6th dose, animals receiving compound A underwent a final day sampling for PK characterization. The data presented above are the summary data for the plasma exposure after IA dose. (a) AUC0-2=area under the plasma concentration-time curve (AUC) from the start of dosing (0) to the last quantifiable time point (τ), which was 2 hours. Expressed in terms of μg/animal.

On day 52, animals received a terminal blood sample and were euthanized. Plasma samples were used for biomarker determination. Urine and synovial fluid was collected at necropsy, and the knees were harvested for histology and pathology analysis.

### Data Analysis and Statistical Methods

Histological processing and sectioning was performed at HistoTox Labs, Inc., and slides were read at Bolder Biopath. Osteoarthritic lesions were scored based on lesion size, depth, severity and changes in subchondral bone and osteophyte formation. Both the femur and the tibia on either side of the meniscal cut were examined. Statistical analysis used Student's two-tailed test or Mann-Whitney U Test (non-parametric).

For terminal plasma samples, biomarker ELISAs were run to quantify levels of PIINP. The kit procedures were followed as per manufacturer's instructions. Data were analyzed with a Student's t-test.

### Results

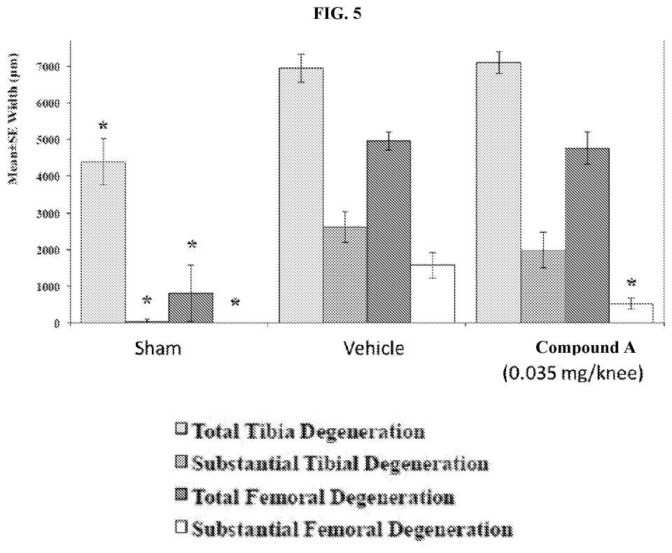

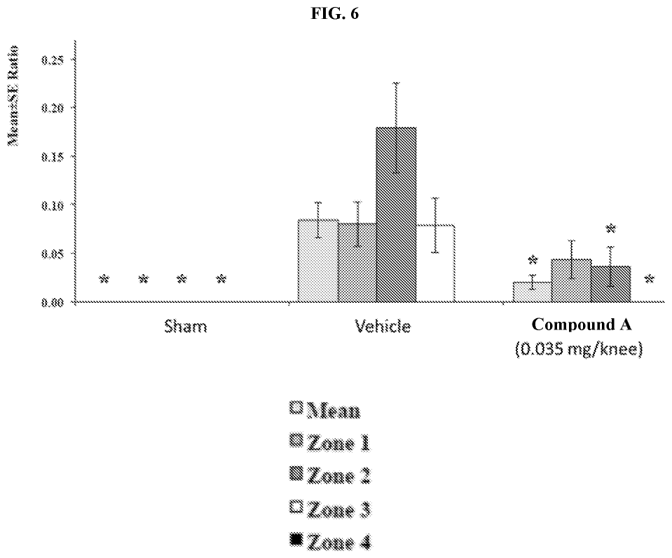

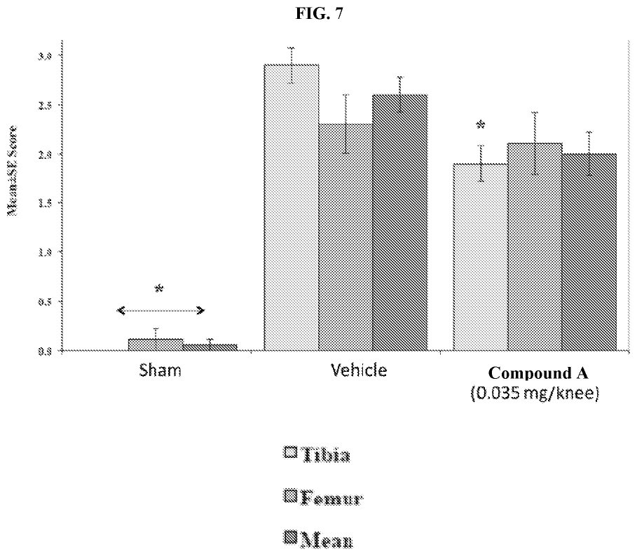

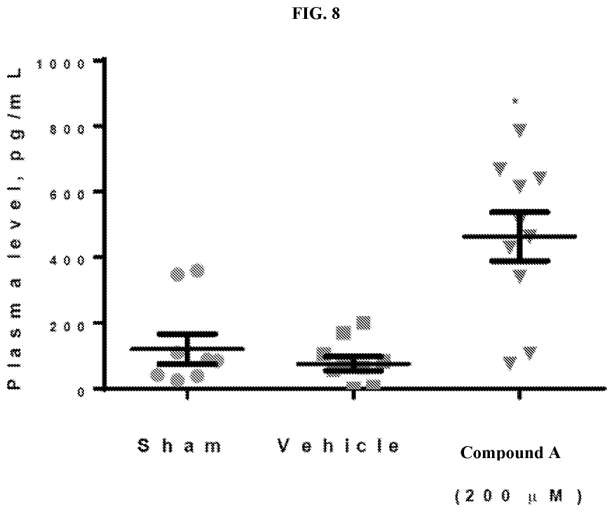

Histological scoring was carried out on a gross morphological scale or by examining aspects of the femur or tibia by either anterior/posterior depth (Levels 1-3) or outer/inner aspects of the tibial plateau or femoral condyle (Zones 1-4). A schematic for this analysis is presented in FIG. 4, where representative vehicle treated knees were stained in India ink post-harvest and photographed to illustrate the lesions present in the dogs. Tibial lesions were examined and assigned a cartilage degeneration score (based on severity) and measured for cartilage lesion depth (expressed as a ratio of the depth of the damaged cartilage to the total depth of the cartilage layer) at various zones and levels (outlined in FIG. 4). Femoral lesions were scored in a similar fashion using pooled data from across several levels (FIG. 5: Female Beagles underwent either sham, or medial meniscal tear surgery to induce osteoarthritis. After once weekly dosing with either vehicle or compound A for 6 weeks, animals were euthanized and knees harvested for histology. Femoral and tibial lesions were measured and scored with substantial femoral degeneration defined as a region within the lesion that shows greater than 50% cartilage loss or damage. The total width of the lesion is plotted and the width of the lesion showing substantial cartilage degeneration is also plotted. n=9-10 Beagles per group. *p<0.05 compared to vehicle and FIG. 6: Female Beagles underwent either sham, or medial meniscal tear surgery to induce osteoarthritis. After weekly dosing with either vehicle or compound A treatment for 6 weeks, animals were euthanized and knees harvested for histology. Femoral lesions were measured using an ocular micrometer. The ratio of the depth of the cartilage lesion to the depth of the total cartilage layer is calculated and plotted above for Zones 1-4. Compound A had a significant effect on decreasing the depth ratio of cartilage lesions in the femur in zones 1, 3 and 4. n=9-10 Beagles per group. *p<0.05 compared to vehicle). Compound A significantly improved the degree of substantial cartilage degeneration in the femur, but showed no effect on the width of total cartilage degeneration (all severities). This indicates that compound A influenced femoral cartilage lesion severity, however did not shrink the overall cartilage size. A more detailed examination of the depth of the femoral lesions showed that compound A treatment was associated with a reduction in the depth of lesions across numerous zones (FIG. 6). In the progression of osteoarthritis, the development of osteophytes is often seen as a compensatory affect to stabilize a joint where lesions are present. Osteophytes are a bony outgrowth on the lateral edges of the tibial plateau. However, the level of bone sclerosis was reduced in animals treated with compound A (FIG. 7: Female Beagles underwent either sham, or medial meniscal tear surgery to induce osteoarthritis over a 7-week study. After once weekly dosing with either vehicle or compound A for 6 weeks, animals were euthanized and knees harvested for histology. Sclerosis of the subchondral bone was scored in the following scale with 0 equal to no sclerosis, 1 (10% of width of femur/tibia had thickened trabeculae), 2 (11-30% of width affected), 3 (31-60% of width affected), 4 (61-90% of width affected) and 5 (>90% of width affected). n=9-10 Beagles per group. *p<0.05 ANOVA compared to vehicle). The remodeling of the subchondral bone (sclerosis) is also viewed as an event characteristic of established or severe osteoarthritis. Plasma exposure was examined after the final dose and compound A was undetectable 1 hour after IA administration (Table 1). Terminal plasma samples were used for biomarker measurements. Among a panel of biomarkers tested, PIINP, a marker for newly synthesized collagen type 2 (derived from the pro-peptide from the N-terminal sequence of collagen type 2), was significantly elevated in dogs treated with compound A (FIG. 8: Female Beagles underwent either sham or medial meniscal tear surgery to induce osteoarthritis over a 7-week study. After once weekly dosing with either vehicle or compound A for 6 weeks, animals were euthanized and terminal blood samples collected (terminal samples were 1 week post final dose). An ELISA was performed to detect circulating levels of PIINP in the plasma. n=9-10 Beagles per group. *p<0.05 ANOVA compared to vehicle).

The goal of this study was to evaluate the efficacy of compound A in a 6-week model of canine osteoarthritis. Compound A is a driver of chondrocyte formation from MSCs. Female Beagles underwent a sham surgery or a partial medial meniscal tear and received weekly doses of either vehicle or compound A via intra-articular injection. All animals were exercised significantly to ensure a robust and uniform lesion formation between dogs. Histological analysis of the knees revealed improvement in those animals receiving compound A treatment. There was a significant reduction in the degree of substantial cartilage degeneration in the femur in animals treated with compound A. Animals treated with compound A appeared, also, to have less severe tibial cartilage damage, although this effect was not statistically significant. These data, in combination with the elevation in circulating levels of collagen marker PIINP, suggest that compound A influenced chondrocyte formation and matrix remodeling/preservation, resulting in a lessening of osteoarthritic outcome. In addition to lesion scoring, peripheral effects secondary to osteoarthritic lesions, such as changes in subchondral bone, also showed beneficial effects upon compound A treatment. Osteophytes (bony projections toward the lateral edges of the tibial plateau) are thought to be compensatory joint stabilizing outgrowths that form secondary to an arthritic lesion. Though potentially beneficial in stabilizing the joint, osteophytes are often a source of pain for OA sufferers and limit joint range of motion. In addition to osteophyte formation, changes in the subchondral bone are also clinically relevant as both can be detected by radiographic measures. Though more characteristic of late stage or severe OA, bone sclerosis is an irreversible change in the subchondral bone, rendering the joint more fragile with significant losses of bone integrity. Compound A treatment improved bone sclerosis, potentially due to the decrease in lesion severity seen in these treated animals. This study shows a significant effect of compound A on the development of osteoarthritis in a surgical model of canine OA. Treated animals show a decrease in lesion depth and severity and decreased bone sclerosis when compared to untreated animals. This shows promise for compound A as a potential treatment for OA.

### Example 13: In Vitro FLNA Binding

### Cell Culture

Primary human MSCs were obtained from Cell Applications. The hMSCs were grown in Mesenchymal Stem Cell expansion media and used between passage 2 and 8 for all experiments. Nuclear cell lysate fractionation and western blotting

hMSCs were treated with compound A at indicated concentrations for 2 hours, washed with icecold PBS once, covered with cell lysis buffer (20 mM HEPES, pH7.9, 10 mM NaCl, 3 mM MgCl2, 0.1% NP-40, 10% glycerol, 0.2 mM EDTA, 1 mM DTT and protease inhibitor cocktail), and incubated on ice for 15 min. The cells were scraped off the dishes mechanically, and pipetted gently to break the cell debris. The cell lysate was centrifuged at 2000 rpm at 4° C. for 5 min and the supernatant was saved as the cytosolic fraction. The pellet was washed with washing buffer (20 mM HEPES, pH 7.9, 20% glycerol, 0.2 mM EDTA, 1 mM DTT and protease inhibitor cocktail) once and centrifuged at 2000 rpm for 5 min. The supernatant was discarded, the pellets were suspended in nuclear extraction buffer (2 mM HEPES, pH7.9, 400 mM NaCl, 20% glycerol, 0.2 mM EDTA, 1 mM DTT and protease inhibitor cocktail) and then incubated on ice for 45 min. The mixture was centrifuged at 13,000 rpm for 15 min at 4° C., and the supernatant was saved as nuclear fraction.

Proteins were resolved using SDS-PAGE gel electrophoresis, transferred to a PVDF membrane using semi-dry blotting cell. The membrane was blocked in blocking buffer (LiCor) for 1 hr at room temperature, followed by incubation with primary antibody in blocking buffer at 4° C. overnight. The membrane was rinsed 3 times with PBST (phosphate buffered saline containing 0.1% triton X-100), incubated with fluorophoreconjugated secondary antibody in blocking buffer for 1 hour at room temperature, rinsed with PBST for at least 3 times, and imaged using Li-Cor Odyssey CLx imaging system.

### Plasmid Construction and Protein Expression

In vitro binding between FLNA FC-1 fragment and the affinity probe was carried out as previously described, with compound A as a competitor at concentration 50 times (25 μM) of that of the probe (0.5 μM).

Compound a Competes with the Biotin-Azide Affinity Probe in Binding to FLNA In Vitro

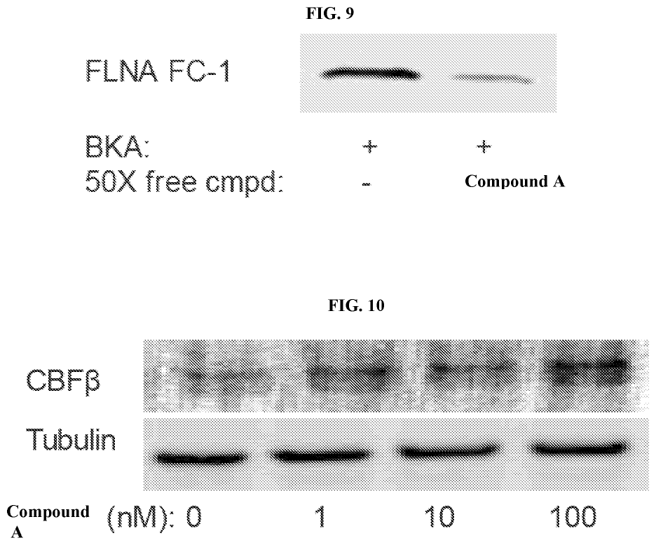

The biological mechanism of kartogenin (KGN) was elucidated through synthesis of a biotin-kartogenin-azide (BKA) affinity probe which was used as a tool to identify the subsequent interaction with FLNA. Subsequently the FC-1 fragment of FLNA was validated to directly mediate the binding of KGN. To determine if compound A retained this functional property, the FLNA FC-1 fragments was incubated with BKA in the absence or presence of compound A. Compound A inhibited the ability of the probe to bind to the FC-1 fragment of FLNA (FIG. 9: The FLNA FC-1 fragment (10 μg/mL) was incubated with 0.5 μM biotin-KGN-azide (BKA) in the absence or presence of 25 μM of compound A at room temperature for 1 hr, photo crosslinked UC 60 nm) for 30 min, and analyzed by Western blotting using anti-biotin antibody).

### Induction of CBFβ Nuclear Localization Through Compound A

Previously KGN was demonstrated to increase the nuclear localization of CBFβ. Furthermore, this nuclear localization was found to be required for KGN to induce chondrogenic differentiation in vitro. CBFβ is a critical transcription co-factor that when bound to RUNX1 can promote hyaline articular cartilage differentiation. To determine if compound A retained this function, hMSCs were incubated with compound A with 1, 10 and 100 nM compound A for 1 hour prior to collection, cell lysis, nuclear fractionation and separation by Western blotting. An increase of CBFβ levels in the nuclear fraction was seen after treatment with compound A at all concentrations tested (FIG. 10: Human MSCs were incubated with compound A at indicated concentrations for 1 hour, nuclear fractions were extracted and analyzed by Western blotting using anti-CBFβ antibody; tubulin (in the same nuclear fractions) was used as a loading control).

Stem or progenitor cells within the articular cartilage support general maintenance and are a possible mediator of tissue repair after injury. Compound A is a low molecular weight clinical candidate which promotes the differentiation of cartilage stem/progenitor cells towards mature, healthy articular chondrocytes and may be beneficial upon direct injection into an affected joint. This report confirms the interaction between compound A and filamin A FC-1 fragment and the subsequent nuclear localization of the chondrogenic transcription co-factor CBFβ. The data confirm that compound A targets the same molecular mechanism as the parent molecule, kartogenin, to promote chondrocyte differentiation.

### Example 14: In Vitro Chondrogenesis of Compound A

### Test System and Experimental Design

Primary human MSCs (40,000) were plated into each well of a 96-well round-bottom cell culture plate, centrifuged briefly to allow cells to aggregate and treated with compound A at the indicated concentration (between 0.1 nM and 10 μM) for 7 days in serum free Dulbecco's Modified Eagle's Medium (DMEM). The RNA was exacted using RNeasy kit following the manufacturer's protocol. cDNA was synthesized using SuperScript III first strand synthesis kit and the transcript levels of proteoglycan 4 (PRG4), sex-determining region Y-Box 9 (50×9) and cartilage-oligo matrix protein (COMP) were determined using Taqman gene expression assays (probes) from ThermoFisher.

### Data Analysis and Statistical Methods

ΔΔCT method was applied to calculate the relative abundance of each gene using (3-actin as normalizing control using ViiA 7 RUO software (ThermoFisher). The abundance of each gene of each sample was expressed as fold over DMSO-treated controls. n=3. Dose response for each marker gene was analyzed using GraphPad Prism 7 software.

### Results

Following treatment of primary human MSCs with compound A in dose response (0-10 μM) for 7 days, isolation of the mRNA, reverse transcription and qPCR, the fold increase was calculated over cells treated with DMSO alone. Over a two-fold increase was documented at 12-1000 nM compound A for PRG4 (EC50˜3.9 nM), a three-fold increase was documented at 12-1000 nM for COMP (EC50˜4.5 nM), and a three to four-fold increase was documented at 4-1000 nM for SOX9 (EC50˜2.2 nM), (Table 12).

TABLE 12
Induction (fold increase) of cartilage-associated
genes after 7-day treatment with compound A

| Compound A (nM) | PRG4 | COMP | SOX9 |
|---|---|---|---|
| 0.457 | 1.082 ± 0.493 | 1.005 ± 0.100 | 1.022 ± 0.231 |
| 1.37 | 0.582 ± 0.513 | 0.776 ± 0.107 | 1.002 ± 0.260 |
| 4.11 | 1.795 ± 0.561 | 1.322 ± 0.804 | 3.296 ± 0.719 |
| 12.3 | 2.226 ± 0.378 | 2.942 ± 0.575 | 2.505 ± 0.242 |
| 37.0 | 1.204 ± 0.360 | 1.356 ± 0.590 | 1.768 ± 0.264 |
| 111 | 1.024 ± 0.700 | 0.706 ± 0.193 | 1.977 ± 0.721 |
| 333 | 2.22 ± 0.960 | 1.896 ± 0.187 | 4.193 ± 0.765 |
| 1000 | 2.134 ± 0.785 | 3.121 ± 0.709 | not done |

Compound A promotes the differentiation of MSCs towards articular chondrocytes. Using quantitative RT-PCR, the data demonstrate the ability of compound A to induce a master transcription factor of chondrocyte differentiation by three to four-fold and two cartilage extracellular proteins (PRG4 and cartilage oligomeric matrix protein) by two-to-three-fold. Together these data confirm the ability of compound A to induce chondrocyte differentiation from progenitor cells.

### Example 15: Pharmakokinetics Studies

### Single Dose Studies

### Example 15a: Intra-Articular Single Dose Pharmacokinetics Study in Rat

Knee joint and plasma exposure were determined in rats (N=2) after a single IA dose (30 μl of a 100 μM solution [(dimethyl sulfoxide/sterile saline vehicle) or 1.05 μg/knee] of compound A. The injected whole knee was harvested immediately after dosing and at 30, 60 and 120 minutes post-dose. Plasma samples were also collected at the same time points. Compound A concentration in the knee immediately after IA injection was 925.5 ng/ml, but fell below the LLQ at 120 minutes. Plasma concentration of compound A was an average AUC_{(0-inf)} of 33.9 (hr*ng/mL) and an average Cₘₐₓ of 53.3 ng/ml and a t_{1/2} of 0.481 hr.

### Example 15b: Intra-Articular Acute Toxicity Study in Crl:CD(SD) Rat

The plasma toxicokinetic profile of compound A was determined after a single IA dose in Crl:CD (SD) male rats. Compound A was given once to male rats (5/group) at concentrations of 0 (vehicle: 1% Ethanol, 10% PEG3350, 0.5% Tween80 in sterile water), 70, 200, or 400 μg/mL, by intra-articular injection (one knee) in a volume of 30 μL. An additional 3 male rats per group were given compound A at 70 or 400 μg/mL for toxicokinetic evaluation. Toxicokinetic parameters were determined, where feasible, by non-compartmental pharmacokinetic analysis. Representative pharmacokinetic parameters are summarized in Table 13. Compound A systemic plasma/serum exposure (mean Cₘₐₓ and AUC₀₋ₜ) increased in a less than dose proportionally. For the dose increment of 5.7 fold (from 2.1 to 12 μg), mean Cₘₐₓ and AUC₀₋ₜ both increased 2-fold. At both doses, plasma Tₘₐₓ occurred at 0.33 hours.

TABLE 13
Representative Mean compound A Toxicokinetic Parameters in Male
Rats administered a Single Intra-articular Dose of compound A.

| | |
|---|---|
| Parameter^{a} | Male Rat |

| Dose (μg/knee) | 2.1^{c} | 12^{c} |
|---|---|---|
| AUC₀₋ₜ ^{b} | 12.1 | 23.9 |
| (ng*h/mL) | [10.3-13.2] | [18.1-27.9] |
| Cₘₐₓ | 13.8 | 27.8 |
| (ng/mL) | [12.9-14.8] | [19.7-33.5] |
| Tₘₐₓ | 0.33 | 0.33 |
| (h) | [0.33-0.33] | [0.33-0.33] |
| Tₗₐₛₜ | 2.00 | 2.00 |
| (h) | [2.00-2.00] | [2.00-2.00] |

^{a}Where applicable, results are reported as mean [range] with the exception of Tₘₐₓ and Tₗₐₛₜ reported as median [range]
^{b} AUC₀₋ₜ = area under the plasma concentration time curve (AUC) from the start of dosing (0) to the last quantifiable time point (t), which was 2 hours unless marked * indicating t = 4 hours
^{c}Expressed in terms of μg/knee

### Example 15c: Intravenous Acute Toxicity Study in Crl:CD(SD) Rat

The plasma TK of compound A was determined after a single IV dose in Crl:CD(SD) rats. Compound A was given once to rats (5/sex/group) at 0 (vehicle), 1 or 2.4 mg/kg by IV slow injection (5 minutes). For toxicokinetic evaluation, an additional 3 rats/sex/group were added for the 1 or 2.4 mg/kg dose groups. Toxicokinetic parameters were determined, where feasible, by non-compartmental analysis. Representative plasma pharmacokinetic parameters are summarized in Table 14. After single slow IV administration (5 min/rat) of compound A at 1 and 2.4 mg/kg, Tₘₐₓ occurred at 0.08 hours, corresponding to the first sample drawn, in both male and female rats. In animals given 2.4 mg/kg, mean Cₘₐₓ (9430 ng/mL in males; 9020 ng/mL in females) was comparable to that in animals receiving 1 mg/kg (11200 ng/mL in males; 9190 ng/mL in females), whereas mean AUC₀₋ₜ was approximately 1.8 fold higher in males and 2.0 fold higher in females dosed at mg/kg compared to those receiving 1 mg/kg. Following single IV administration of compound A, T_{1/2} ranged from approximately 0.3 to 0.6 hours. Overall, no notable gender differences in compound A systemic exposure (Cₘₐₓ and AUC₀₋ₜ) were observed in either dosing regimen.

TABLE 14
Representative compound A Mean Toxicokinetic Parameters
Rats Administered a Single Intravenous Dose of compound A

| Parameter^{a} | Male | | Female | |
|---|---|---|---|---|
| Dose (mg/kg) | 1 | 2.4 | 1 | 2.4 |
| AUC₀₋ₜ ^{b} | 2500 | 4510 | 2900 | 5690* |
| (ng*h/mL) | [1990-3170] | [3800-5800*] | [2180-3750] | [5240-6580]* |
| Cₘₐₓ | 11200 | 9430 | 9190 | 9020 |
| (ng/mL) | [4570-19100] | [7150-10700] | [5160-17200] | [7740-11300] |
| Tₘₐₓ | 0.08 | 0.08 | 0.08 | 0.08 |
| (h) | [0.08-0.08] | [0.08-0.08] | [0.08-0.08] | [0.08-0.08] |
| Tₗₐₛₜ | 2 | 2 | 2 | 4 |
| | [2-2] | [2-4] | [2-4] | [4-4] |
| t_{1/2} (hours) | 0.37 | 0.40 | 0.48 | 0.53 |
| | [0.28-0.46] | [0.38-0.45] | [0.41-0.55] | [0.51-0.56] |

^{a}Where applicable, results are reported as mean [range] with the exception of Tₘₐₓ and Tₗₐₛₜ reported as median [range]
^{b} AUC₀₋ₜ = area under the plasma concentration-time curve (AUC) from the start of dosing (0) to the last quantifiable time point (t), which was 2 hours unless marked * indicating t = 4 hours

### Example 15d: Intra-Articular Acute Toxicity Study in the Beagle Dog

The plasma and synovial fluid toxicokinetic profile of compound A was determined after a single IA dose in male beagle dogs. Compound A was given once to dogs (2 males/group); each animal received 2 injections, one at concentrations of 0 (vehicle) in the right knee and one at concentrations of 70, 200 or 400 ng/mL, equivalent to a total of 35, 100 and 200 μg/dog knee, in the contralateral knee (left) at a constant volume of 500 μL. Toxicokinetic parameters were determined by non-compartmental pharmacokinetic analysis Representative pharmacokinetic parameters are summarized in Table 15. Compound A concentrations in synovial fluid samples at study termination were all below the LLQ (0.5 ng/ml). Compound A systemic plasma exposure (mean Cₘₐₓ and AUC₀₋ₜ) increased in a less than dose-proportional manner. For the dose increment of 2.9 fold (from 35 to 100 ng, corresponding to 70 and 200 ng/mL, respectively), mean Cₘₐₓ and AUC₀₋ₜ both increased 0.7 fold; for the dose increment of 5.7 fold (from 35 to 200 ng, corresponding to 70 and 400 ng/mL, respectively), mean Cₘₐₓ and AUC₀₋ₜ both increased approximately 1.5 fold. At doses compound A of 35, 100 and 200 ng, plasma Tₘₐₓ ranged from 0.08 to 1 hours.

TABLE 15
Summary of Mean Toxicokinetic Parameters in Plasma Following
a Single Intra-articular Dose to the Beagle Dog.

| Dose | Animal | Cₘₐₓ | Tₘₐₓ | AUC₀₋ₜ^{a} | Tₗₐₛₜ |
|---|---|---|---|---|---|
| (μg/knee)^{b} | Number | (ng/mL) | (h) | (ng*h/mL) | (h) |
| 35 | 41M | 2.14 | 0.33 | 5.21 | 4.00 |
| | 42M | 2.21 | 0.08 | 5.41 | 4.00 |
| | Mean | 2.17 | | 5.31 | 4.00 |
| 100 | 43M | 4.43 | 1.00 | 13.0 | 4.00 |
| | 44M | 4.75 | 0.33 | 11.7 | 4.00 |
| | Mean | 4.59 | | 12.3 | 4.00 |
| 200 | 45M | 2.81 | 1.00 | 8.18 | 4.00 |
| | 46M | 3.21 | 0.08 | 9.60 | 4.00 |
| | Mean | 3.01 | | 8.89 | 4.00 |

^{a}AUC₀₋ₜ = area under the plasma concentration-time curve (AUC) from the start of dosing (0) to the last quantifiable time point (t), which was 4 hours
^{b}Expressed in terms of μg/knee

### Multiple Dose Studies

### Example 15e: Five Week Intra-Articular Repeat Toxicity Study in CD Rat with a 14 Day Recovery Period

The plasma toxicokinetic profile of compound A in Crl:CD(SD) rats was determined in a multiple dose IA toxicity study. Compound A was given to rats (13/sex/group), once a week, for 5 weeks, at nominal concentrations of 0 (vehicle), 70, 140 and 200 μg/mL/week, at a fixed volume of 30 μL, corresponding to 7, 14 and 20 μg/kg/week (0, 2.1, 4.2 or 6 μg/knee). The site of injection was the right femoro-tibial joint for each dose. Three rats/sex/group were evaluated for toxicokinetics. Toxicokinetic analysis was performed by non-compartmental pharmacokinetic analysis. All computations utilized the nominal sampling times. Representative pharmacokinetic parameters are summarized in Table 16. Plasma samples collected from vehicle-control animals, sampled at 1 hour after the start of dosing on both Day 1 and Day 29, were analyzed. In all samples, concentrations of compound A were below the LLQ of 0.25 ng/mL. Following single (Day 1) and repeat (Day 29) once a week IA administration of compound A to male and female rats, compound A was quantifiable in the plasma of all animals up to at least 2 hours from the start of dosing. Tₘₐₓ as median value generally occurred at 0.33 hours after dosing in both male and female rats at all dose levels evaluated, with the exception of 7 μg/kg/week on Day 29 in females where it was 1 hour. Following single (Day 1) and repeat (Day 29) once a week IA administration, plasma systemic exposure to compound A (mean Cₘₐₓ and mean AUC₀₋ₜ) increased proportionally with increasing dose in both male and female rats. On Day 1, for a dose increment of 2.9-fold (from 7 to 20 μg/kg/week) there was an approximate 4.1 (male) and 3.1 (female)-fold increase in mean Cₘₐₓ; 4.4 (male) and 3.2 (female)-fold increase in mean AUC₀₋ₜ, respectively, whereas on Day 29 mean Cₘₐₓ increased about 2.7 (male) and 3.2 (female)-fold and the mean AUC₀₋ₜ, increased 2.7 (male) and 2.1 (female)-fold, respectively. Generally no significant (where significance is considered to be >2-fold) differences in compound A systemic exposure were observed following single and repeat IA administration across the entire dose range evaluated in both genders.

TABLE 16
Representative Mean compound A Plasma Toxicokinetic Parameters in
Rats Administered Five Weekly Intra-articular Doses of compound A

| | | Dose of compound A (μg/kg/week)^{b} or μg/knee | | | | | |
|---|---|---|---|---|---|---|---|
| | | 70 μg/kg/week = 2.1 μg/knee | | 140 μg/kg/week = 4.2 μg/knee | | 200 μg/kg/week = 6 μg/knee | |
| Parameter^{a} | Day | Males | Females | Males | Females | Males | Females |
| AUC₀₋ₜ ^{c} | 1 | 8.14 | 20.3 | 18.4 | 52.3 | 35.5 | 64.9 |
| (ng*h/mL) | | [5.37-10.4] | [16.1-26.2] | [15.5-22.7] | [44.7-64.8] | [29.9-46.7] | [34.2-111] |
| | 29 | 8.33 | 18.5 | 15.4 | 43.5 | 22.9 | 39.3 |
| | | [5.72-10.0] | [15.7-22.5] | [13.4-19.0] | [33.0-55.6] | [15.7-32.0] | [18.6-60.8] |
| Cₘₐₓ | 1 | 8.24 | 19.5 | 23.5 | 42.3 | 33.9 | 59.8 |
| (ng/mL) | | [4.12-11.3] | [15.7-24.9] | [19.1-31.4] | [41.2-42.9] | [20.3-51.1] | [34.7-100] |
| | 29 | 8.41 | 11.6 | 18.6 | 34.5 | 22.9 | 36.6 |
| | | [6.30-11.3] | [10.9-12.3] | [14.5-24.2] | [30.2-36.8] | [19.6-27.1] | [25.5-48.0] |
| Tₘₐₓ (h) | 1 | 0.33^{d} | 0.33^{d} | 0.33 | 0.33^{d} | 0.33 | 0.33^{d} |
| | | | | [0.08-0.33] | | [0.08-0.33] | |
| | 29 | 0.33^{d} | 1.00 | 0.33^{d} | 0.33^{d} | 0.33^{d} | 0.33^{d} |
| | | | [0.33-1.00] | | | | |

^{a}Where applicable, results are reported as mean [range] with the exception of Tₘₐₓ reported as median [range]
^{b}Nominal dose levels are given in terms of total once a week dose of the parent test item. Dose was calculated on the bases of mean body weight (300 g) for 30 μL administration per rat
^{c} AUC₀₋ₜ = area under the plasma concentration-time curve (AUC) from the start of dosing (0) to the last quantifiable time point (t)
^{d}Range is not reported since minimum and maximum values are identical

### Example 15f: Five Week Intravenous Repeat Toxicity Study in CD Rat with a 14 Day Recovery Period

The plasma toxicokinetic profile of compound A in Crl:CD(SD) rats was determined in a multiple IV toxicity study. Compound A was given to rats (10/sex/group) at 0, 0.25, 0.75, or 2.5 mg/kg once weekly for 5 weeks by intravenous (slow bolus) injection at a dose volume of 10 mL/kg. Additional animals (3/sex/group) were given compound A at 0 (vehicle), 0.25, 0.75, or 2.5 mg/kg/week for toxicokinetic evaluation. Toxicokinetic analysis was performed by non-compartmental pharmacokinetic analysis. All computations utilized the nominal sampling times. Representative pharmacokinetic parameters are summarized in Table 17. Plasma samples collected from vehicle-control animals, sampled at 0.33 hours (=20 minutes) from the start of dosing on both Day 1 and Day 29, were analyzed. In all samples, concentrations of compound A were below the LLQ of 0.25 ng/mL. Following single (Day 1) and repeated (Day 29) once a week IV administration of compound A in male and female rats, compound A was quantifiable in the plasma of all animals up to at least 4 hours from the start of dosing. Tₘₐₓ occurred at 0.08 hours from the start of infusion, corresponding to the first sample drawn in both male and female rats at all doses evaluated. compound A VA, where calculated, ranged from approximately 0.36 to 0.84 hours at all doses evaluated in both genders. After single (Day 1) and repeated (Day 29) once a week IV administration plasma systemic exposure of compound A (either in terms of mean Cₘₐₓ and mean AUC₀₋ₜ), increased generally with no major deviation from proportionality up to 0.75 mg/kg/week in both genders with the exception in male rats on Day 29 where over-proportionality was observed for Cₘₐₓ. Overall for a dose increment of 10-fold (from 0.25 to 2.5 mg/kg/week) plasma exposure of compound A increased in under-proportionally on Day 1. hereas on Day 29, both mean Cₘₐₓ and mean AUC₀₋ₜ generally increased proportionally with increasing dose in both male and female rats. Generally no significant (where significance is considered to be >2 fold) differences in compound A systemic exposure were observed following single and repeated IV administration across the entire dose range evaluated in both genders. Overall, no significant gender differences in compound A mean Cₘₐₓ and mean AUC₀₋ₜ were observed on either Day 1 or 29 across the dose range

TABLE 17
Representative Mean Plasma compound A Toxicokinetic Parameters in
Rats Administered Five Weekly Intravenous Doses of compound A

| | | Dose of compound A (mg/kg/week)^{b} | | | | | |
|---|---|---|---|---|---|---|---|
| | | 0.25 | | 0.75 | | 2.5 | |
| Parameter^{a} | Day | Male | Females | Male | Females | Male | Females |
| AUC₀₋ₜ ^{c} | 1 | 633 | 764 | 2160 | 1920 | 3020 | 2810 |
| (ng*h/mL) | | [298-986] | [744-803] | [1960-2320] | [1800-2030] | [2110-3980] | [2240-3170] |
| | 29 | 657 | 871 | 3120 | 2250 | 9410 | 9320 |
| | | [578-772] | [769-1000] | [1810-5250] | [2040-2520] | [7780-11000] | [7840-11000] |
| Cₘₐₓ | 1 | 1940 | 1230 | 5360 | 3490 | 6070 | 5790 |
| (ng/mL) | | [821-3520] | [1030-1460] | [3950-7870] | [3290-3770] | [4420-7780] | [4780-7260] |
| | 29 | 2060 | 1380 | 13800 | 4800 | 15400 | 30200 |
| | | [1340-3070] | [1200-1480] | [3910-33200] | [3690-5930] | [13600-17300] | [14300-58800] |
| Tₘₐₓ (h) | 1 | 0.08^{a} | 0.08^{d} | 0.08^{d} | 0.08^{d} | 0.08^{d} | 0.08^{d} |
| | 29 | 0.08^{d} | 0.08^{d} | 0.08^{d} | 0.08^{d} | 0.08^{d} | 0.08^{d} |
| t_{1/2} | 1 | 0.39 | 0.58 | 0.63 | 0.59 | 0.64 | 0.63 |
| | | [0.36-0.42] | [0.47-0.73] | [0.60-0.66] | [0.44-0.67] | [0.60-0.68] | [0.61-0.66] |
| | 29 | 0.42 | 0.54 | 0.43 | 0.48 | 0.71 | 0.73 |
| | | [0.39-0.47] | [0.49-0.57] | [0.40-0.45] | [0.44-0.53] | [0.61-0.81] | [0.62-0.84] |

^{a}Where applicable, results are reported as mean [range] with the exception of Tₘₐₓ reported as median [range]
^{b}Nominal dose levels are given in terms of total once a week dose of the free base
^{c} AUC₀₋ₜ = area under the plasma concentration-time curve (AUC) from the start of dosing (0) to the last quantifiable time point (t)
^{d}Range is not reported since minimum and maximum values are identical.

### Example 15g: Five Week Intra-Articular Repeat Toxicity Study in the Beagle Dog with a 14 Day Recovery Period

The plasma and synovial fluid toxicokinetic profile of compound A in beagle dogs was determined in a multiple dose IA toxicity study. Compound A was given to dogs (right knee; 3/sex/group) once a week, for 5 weeks, at nominal concentrations of 0, 70, 140 and 200 μg/mL, at a fixed volume of 500 μL (thus giving nominal doses of 0, 35, 70 and 100 μg/knee/week). Toxicokinetic analysis was performed by non-compartmental analysis. All computations utilized the nominal sampling times. Representative plasma pharmacokinetic parameters are summarized in Table 18. Plasma samples collected from vehicle control animals, sampled at the same timepoints on Day 1 and Day 29 as compound A-treated animals, were analysed only at expected Tₘₐₓ for compound A (1 hour). In all samples analysed, concentrations of test item were below the lower limit of quantification, which was 0.25 ng/mL. After IA administration of compound A at 3.5, 7.0 and 10 μg/kg/week, compound A was quantifiable in the plasma of all animals up to at least 3 hours after dosing (LLQ=0.25 ng/ml). Tₘₐₓ on Day 1 and Day 29 generally ranged from 0.33 to 1 hour in both male and female dogs. compound A systemic exposure (mean Cₘₐₓ and AUC₀₋ₜ) on both Day 1 and Day 29 increased approximately dose proportionally. No significant differences (where significance is considered to be >2-fold) in compound A systemic exposure were observed following single and repeat IA administration across the entire dose range. Overall, no significant gender differences in compound A systemic exposure (Cₘₐₓ and AUC₀₋ₜ) were observed on Day 1 and Day 29 across the dose range. In all synovial fluid samples, which were collected at study termination, concentrations of test item were below the LLQ (20 ng/mL).

TABLE 18
Representative Mean Plasma compound A Toxicokinetic
Parameters in Dogs administered Five Weekly
Intra-articular Doses of compound A

| Parameter^{a} | | | | |
|---|---|---|---|---|
| AUC₀₋ₜ ^{c} | | Dose of compound A (μg/knee)^{b} | | |
| (ng*h/mL) | Day | 35 | 70 | 100 |

Male

| | | | | |
|---|---|---|---|---|
| | 1 | 8.60 | 13.8 | 21.9 |
| | | [7.68-9.98] | [11.2^{#}-18.8] | [14.8^{#}-28.7] |
| | 29 | 8.54 | 12.2^{#} | 17.0 |
| | | [8.33^{#}-8.83] | [11.6-13.1]^{#} | [15.5-18.5] |
| Cₘₐₓ (ng/mL) | 1 | 3.49 | 5.59 | 8.40 |
| | | [2.55-4.55] | [5.36-5.75] | [6.26-11.3] |
| | 29 | 4.42 | 5.64 | 5.63 |
| | | [3.56-4.95] | [5.48-5.83] | [4.11-7.70] |
| Tₘₐₓ (h) | 1 | 0.33 | 0.33 | 1.00 |
| | | [0.33-1.00] | [0.33-1.00] | [1.00-1.00] |
| | 29 | 0.33 | 0.33 | 1.00 |
| | | [0.33-0.33] | [0.08-0.33] | [0.33-2.00] |

Female

| | | | | |
|---|---|---|---|---|
| AUC₀₋ₜ ^{c} | 1 | 9.05 | 21.4 | 26.3 |
| (ng*h/mL) | | [6.74^{#}-11.8] | [17.3-25.9] | [22.3^{#}-28.6] |
| | 29 | 12.6 | 19.4 | 28.7 |
| | | [11.4-13.8] | [16.4-22.0] | [26.3-30.1] |
| Cₘₐₓ (ng/mL) | 1 | 3.72 | 8.36 | 10.6 |
| | | [3.50-4.14] | [6.40-10.8] | [10.3-11.0] |
| | 29 | 4.79 | 6.85 | 10.3 |
| | | [3.87-5.54] | [6.24-7.44] | [8.14-13.0] |
| Tₘₐₓ (h) | 1 | 0.33 | 0.33 | 0.33 |
| | | [0.33-1.00] | [0.33-0.33] | [0.33-1.00] |
| | 29 | 0.33 | 0.33 | 0.33 |
| | | [0.33-1.00] | [0.33-1.00] | [0.33-0.33] |

^{a}Where applicable, results are reported as mean [range] with the exception of Tₘₐₓ reported as median [range]
^{b}Nominal dose levels are given in terms of total weekly dose
^{c} AUC₀₋ₜ = area under the plasma concentration-time curve (AUC) from the start of dosing (0) to the last quantifiable time point (t), which was 6 hours unless marked ^{#} indicating Tₗₐₛₜ = 3 hours

### Example 15h: Five Week Intravenous Repeat Toxicity Study in the Beagle Dog with a 14 Day Recovery Period

The plasma toxicokinetic profile of compound A in Beagle dogs was determined in a repeat dose IV toxicity study compound A was given to dogs (3/sex/group) at 0, 0.125, 0.600 and 1.250 mg/kg/week by IV administration at a dose volume of 5 mL/kg and with an injection rate of 5 mL/min. Toxicokinetic analysis was performed by non-compartmental pharmacokinetic analysis. All computations utilized the nominal sampling times. Representative pharmacokinetic parameters are summarized in Table 19 and Table 20. In all samples analyzed from animals given vehicle, concentrations of test item were below the LLQ (0.25 ng/mL). Following single IV administration of compound A to male and female dogs at 0.125, 0.600, and 1.250 mg/kg/week, compound A was quantifiable in the plasma of all animals up to at least 4 hours after dosing and up to at least 12 hours after dosing when dosed at 1.250 mg/kg (LLQ=0.25 ng/ml). After IV administration of compound A at 0.125, 0.600 and 1.250 mg/kg/week, Tₘₐₓ on Day 1 and Day 29 generally occurred at 0.17 hours (10 minutes), which corresponds to the first sample drawn after dosing, in both male and female dogs. The half-life (t_{1/2}), plasma clearance (Cl) and volume of distribution of compound A at the steady state (V_{SS}) were similar on the first day and after five weeks of once weekly administration (Day 29) of compound A. The mean apparent half-life on Days 1 and 29 ranged from 0.64 to 0.93 hours in both male and female dogs. The average plasma clearance (Cl) ranged from 435 mL/h/kg to 686 mL/h/kg in male dogs and from 492 to 986 mL/h/kg in female dogs. The mean volume of distribution ranged from 440 to 692 mL/kg in male dogs and from 534 to 855 mL/kg in female dogs. Compound A systemic exposure (mean Cₘₐₓ and AUC₀₋ₜ) on both Day 1 and Day 29 increased in a dose proportional manner. In general, no significant differences (where significance is considered to be >2 fold) in compound A systemic exposure were observed following single and repeat weekly IV administration across the entire dose range. Overall no significant gender differences in compound A systemic exposure (Cₘₐₓ and AUC₀₋ₜ) were observed on either Day 1 or Day 29 across the dose range.

TABLE 19
Representative Mean Plasma compound A Toxicokinetic
Parameters in Male Dogs Administered Five Weekly
Intravenous Doses of compound A

| | | Male | | |
|---|---|---|---|---|
| | | Dose of compound A (mg/kg/week)^{b} | | |
| Parameter^{a} | Day | 0.125 | 0.600 | 1.250 |
| AUC ^{c}0-t | 1 | 211 | 1400 | 2510 |
| (ng · h/mL) | | [197^{#}-221^{#}] | [1270^{#}-1650^{#}] | [2030^{#}-2850] |
| | 29 | 181 | 1190 | 2600 |
| | | [158^{#}-197^{#}] | [1120^{#}-1250^{#}] | [1550^{#}-3210] |
| Cₘₐₓ (ng/mL) | 1 | 308 | 2100 | 2580 |
| | | [169-421] | [1120-2840] | [2310-2800] |
| | 29 | 197 | 1140 | 2150 |
| | | [184-207] | [1010-1260] | [1740-2450] |
| Tₘₐₓ (h) | 1 | 0.17 | 0.17 | 0.17 |
| | | [0.17-0.17] | [0.17-0.17] | [0.17-0.17] |
| | 29 | 0.17 | 0.17 | 0.17 |
| | | [0.17-0.17] | [0.17-0.17] | [0.17-0.33] |

^{a}Where applicable, results are reported as mean [range] with the exception of Tₘₐₓ reported as median [range]
^{b}Dose levels are expressed as total weekly dose of the free base.
^{c} AUC₀₋ₜ = area under the plasma concentration-time curve (AUC) from the start of dosing (0) to the last quantifiable time point (t), which was 12 hours unless marked ^{#} indicating Tₗₐₛₜ = 8 hours or 4 hours

TABLE 20
Representative Mean Plasma compound A Toxicokinetic
Parameters in Female Dogs Administered Five
Weekly Intravenous Doses of compound A

| | | Female | | |
|---|---|---|---|---|
| | | Dose of compound A (mg/kg/week)^{b} | | |
| Parameter^{a} | Day | 0.125 | 0.600 | 1.250 |
| AUC ^{c}0-t | 1 | 131 | 1230 | 2300 |
| (ng · h/mL) | | [102^{#}-172^{#}] | [1100^{#}-1380^{#}] | [1870^{#}-2520] |
| | 29 | 178 | 1200 | 2150 |
| | | [143^{#}-199] | [1130^{#}-1240^{#}] | [1570^{#}-2720] |
| Cₘₐₓ (ng/mL) | 1 | 181 | 1330 | 2460 |
| | | [161-199] | [1210-1520] | [2200-2610] |
| | 29 | 209 | 1190 | 2480 |
| | | [187-253] | [1150-1230] | [2140-2760] |
| Tₘₐₓ (h) | 1 | 0.17 | 0.17 | 0.17 |
| | | [0.17-0.17] | [0.17-0.17] | [0.17-0.17] |
| | 29 | 0.17 | 0.17 | 0.17 |
| | | [0.17-0.17] | [0.17-0.17] | [0.17-0.17] |

^{a}Where applicable, results are reported as mean [range] with the exception of Tₘₐₓ reported as median [range]
^{b}Dose levels are expressed as total weekly dose of the free base.
^{c} AUC₀₋ₜ = area under the plasma concentration-time curve (AUC) from the start of dosing (0) to the last quantifiable time point (t)), which was 12 hours unless marked ^{#} indicating Tₗₐₛₜ = 8 hours or 4 hour

What is claimed is:

1. A method for ameliorating arthritis or joint injury in a subject, the method comprising administering to the joint space of a knee of the subject from about 10 μg to about 1000 μg of N-(4-(2-methoxyethyl)phenyl)-2-(methylsulfonamido)benzamide, or a pharmaceutically acceptable salt, or solvate thereof.

2. The method of claim 1, comprising administering to the joint space of a knee from about 10 μg to about 400 μg of N-(4-(2-methoxyethyl)phenyl)-2-(methylsulfonamido)benzamide, or a pharmaceutically acceptable salt, or solvate thereof.

3. The method of claim 1, comprising administering to the joint space of a knee from about 50 μg to about 400 μg of N-(4-(2-methoxyethyl)phenyl)-2-(methylsulfonamido)benzamide, or a pharmaceutically acceptable salt, or solvate thereof.

4. A method for ameliorating arthritis or joint injury in a subject, the method comprising administering to the joint space of a knee of the subject no more than about 1000 μg of N-(4-(2-methoxyethyl)phenyl)-2-(methylsulfonamido)benzamide, or a pharmaceutically acceptable salt, or solvate thereof.

5. The method of claim 4, comprising administering to the joint space of a knee no more than about 400 μg of N-(4-(2-methoxyethyl)phenyl)-2-(methylsulfonamido)benzamide, or a pharmaceutically acceptable salt, or solvate thereof.

6. A method for inducing differentiation of mesenchymal stem cells into chondrocytes in a subject, the method comprising administering to the joint space of a knee of the subject from about 10 μg to about 1000 μg of N-(4-(2-methoxyethyl)phenyl)-2-(methylsulfonamido)benzamide, or a pharmaceutically acceptable salt, or solvate thereof.

7. The method of claim 6, comprising administering to the joint space of a knee from about 10 μg to about 400 μg of N-(4-(2-methoxyethyl)phenyl)-2-(methylsulfonamido)benzamide, or a pharmaceutically acceptable salt, or solvate thereof.

8. The method of claim 6, comprising administering to the joint space of a knee from about 50 μg to about 400 μg of N-(4-(2-methoxyethyl)phenyl)-2-(methylsulfonamido)benzamide, or a pharmaceutically acceptable salt, or solvate thereof.

9. A method for inducing differentiation of mesenchymal stem cells into chondrocytes in a subject, the method comprising administering to the joint space of a knee of the subject not more than about 1000 μg of N-(4-(2-methoxyethyl)phenyl)-2-(methylsulfonamido)benzamide, or a pharmaceutically acceptable salt, or solvate thereof.

10. The method of claim 9, comprising administering to the joint space of a knee no more than about 400 μg of N-(4-(2-methoxyethyl)phenyl)-2-(methylsulfonamido)benzamide, or a pharmaceutically acceptable salt, or solvate thereof.

11. The method of any of claims 1-10, wherein N-(4-(2-methoxyethyl)phenyl)-2-(methylsulfonamido)benzamide, or a pharmaceutically acceptable salt, or solvate thereof is administered to the subject annually.

12. The method of any of claims 1-10, wherein N-(4-(2-methoxyethyl)phenyl)-2-(methylsulfonamido)benzamide, or a pharmaceutically acceptable salt, or solvate thereof is administered to the subject every eleven months.

13. The method of any of claims 1-10, wherein N-(4-(2-methoxyethyl)phenyl)-2-(methylsulfonamido)benzamide, or a pharmaceutically acceptable salt, or solvate thereof is administered to the subject every ten months.

14. The method of any of claims 1-10, wherein N-(4-(2-methoxyethyl)phenyl)-2-(methylsulfonamido)benzamide, or a pharmaceutically acceptable salt, or solvate thereof is administered to the subject every nine months.

15. The method of any of claims 1-10, wherein N-(4-(2-methoxyethyl)phenyl)-2-(methylsulfonamido)benzamide, or a pharmaceutically acceptable salt, or solvate thereof is administered to the subject every eight months.

16. The method of any of claims 1-10, wherein N-(4-(2-methoxyethyl)phenyl)-2-(methylsulfonamido)benzamide, or a pharmaceutically acceptable salt, or solvate thereof is administered to the subject every seven months.

17. The method of any of claims 1-10, wherein N-(4-(2-methoxyethyl)phenyl)-2-(methylsulfonamido)benzamide, or a pharmaceutically acceptable salt, or solvate thereof is administered to the subject every six months.

18. The method of any of claims 1-10, wherein N-(4-(2-methoxyethyl)phenyl)-2-(methylsulfonamido)benzamide, or a pharmaceutically acceptable salt, or solvate thereof is administered to the subject every five months.

19. The method of any of claims 1-10, wherein N-(4-(2-methoxyethyl)phenyl)-2-(methylsulfonamido)benzamide, or a pharmaceutically acceptable salt, or solvate thereof is administered to the subject every four months.

20. The method of any of claims 1-10, wherein N-(4-(2-methoxyethyl)phenyl)-2-(methylsulfonamido)benzamide, or a pharmaceutically acceptable salt, or solvate thereof is administered to the subject every three months.

21. The method of any of claims 1-10, wherein N-(4-(2-methoxyethyl)phenyl)-2-(methylsulfonamido)benzamide, or a pharmaceutically acceptable salt, or solvate thereof is administered to the subject every two months.

22. The method of any of claims 1-10, wherein N-(4-(2-methoxyethyl)phenyl)-2-(methylsulfonamido)benzamide, or a pharmaceutically acceptable salt, or solvate thereof is administered to the subject monthly or weekly.

23. The method of any of claims 1-22, wherein about 25 μg of N-(4-(2-methoxyethyl)phenyl)-2-(methylsulfonamido)benzamide, or a pharmaceutically acceptable salt, or solvate thereof is administered.

24. The method of any of claims 1-22, wherein about 50 μg of N-(4-(2-methoxyethyl)phenyl)-2-(methylsulfonamido)benzamide, or a pharmaceutically acceptable salt, or solvate thereof is administered.

25. The method of any of claims 1-22, wherein about 100 μg of N-(4-(2-methoxyethyl)phenyl)-2-(methylsulfonamido)benzamide, or a pharmaceutically acceptable salt, or solvate thereof is administered.

26. The method of any of claims 1-22, wherein about 150 μg of N-(4-(2-methoxyethyl)phenyl)-2-(methylsulfonamido)benzamide, or a pharmaceutically acceptable salt, or solvate thereof is administered.

27. The method of any of claims 1-22, wherein about 200 μg of N-(4-(2-methoxyethyl)phenyl)-2-(methylsulfonamido)benzamide, or a pharmaceutically acceptable salt, or solvate thereof is administered.

28. The method of any of claims 1-22, wherein about 250 μg of N-(4-(2-methoxyethyl)phenyl)-2-(methylsulfonamido)benzamide, or a pharmaceutically acceptable salt, or solvate thereof is administered.

29. The method of any of claims 1-22, wherein about 300 μg of N-(4-(2-methoxyethyl)phenyl)-2-(methylsulfonamido)benzamide, or a pharmaceutically acceptable salt, or solvate thereof is administered.

30. The method of any of claims 1-22, wherein about 350 μg of N-(4-(2-methoxyethyl)phenyl)-2-(methylsulfonamido)benzamide, or a pharmaceutically acceptable salt, or solvate thereof is administered.

31. The method of any of claims 1-22, wherein about 400 μg of N-(4-(2-methoxyethyl)phenyl)-2-(methylsulfonamido)benzamide, or a pharmaceutically acceptable salt, or solvate thereof is administered.

32. The method of any of claims 1-31, wherein N-(4-(2-methoxyethyl)phenyl)-2-(methylsulfonamido)benzamide, or a pharmaceutically acceptable salt, or solvate thereof is administered in a volume of from about 1 mL to about 5 mL.

33. The method of any of claims 1-31, wherein N-(4-(2-methoxyethyl)phenyl)-2-(methylsulfonamido)benzamide, or a pharmaceutically acceptable salt, or solvate thereof is administered in a volume about or no more than about 5 mL.
